SEQUENCE LISTING (63 sequences)

SEQ ID NO: 1 - protein, 90 aa, Artificial Sequence
  “N” apoferritin
MSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 2 - protein, 85 aa, Artificial Sequence
  “C” apoferritin
GKTPDAMKAAMALEKKLNQALLDLHALGSARTDPHLCDFLETHFLDEEVKLIKKMGDHLTNLHRLGGPEAGLGEYLFERLTLRHD

SEQ ID NO: 3 - protein, 85 aa, Artificial Sequence
  “C” apoferritin
GKTPDAMKAAMALEKKLNQALLDLHALGSARTDPHLCDFLETHFLDEEVKLIKKMGDHLTNLHRLGGPEAGLGEYLFERLTLKHD

SEQ ID NO: 4 - protein, 27 aa, Artificial Sequence
  Linker
GGGGSGGGGSGGGGSGGGGSGGGGSGG

SEQ ID NO: 5 - protein, 26 aa, Artificial Sequence
  Linker
GGSGGSGGSGGSGGGSGGSGGSGGSG

SEQ ID NO: 6 - protein, 70 aa, Artificial Sequence
  Linker
GGGGSGGGGSGGGGSGGGGSGGGGSGGGGSGGGGSGGGGSGGGGSGGGGSGGGGSGGGGSGGGGSGGGGS

SEQ ID NO: 7 - protein, 227 aa, Artificial Sequence
  Fc chain 1
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 8 - protein, 227 aa, Artificial Sequence
  Fc chain 2
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 215 aa, Artificial Sequence
  Conatumumab light chain
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 225 aa, Artificial Sequence
  Conatumumab Fab heavy chain
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 11 - protein, 175 aa, Artificial Sequence
  Ferritin
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 12 - protein, 713 aa, Artificial Sequence
  Conatumumab-hFerr
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 13 - protein, 712 aa, Artificial Sequence
  Conatumumab-hFerr
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 14 - protein, 707 aa, Artificial Sequence
  Tigatuzumab-hFerr
DIQMTQSPSSLSASVGDRVTITCKASQDVGTAVAWYQQKPGKAPKLLIYWASTRHTGVPSRFSGSGSGTDFTLTISSLQPEDFATYYCQQYSSYRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSEVQLVESGGGLVQPGGSLRLSCAASGFTFSSYVMSWVRQAPGKGLEWVATISSGGSYTYYPDSVKGRFTISRDNAKNTLYLQMNSLRAEDTAVYYCARRGDSMITTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 15 - protein, 706 aa, Artificial Sequence
  Tigatuzumab-hFerr
DIQMTQSPSSLSASVGDRVTITCKASQDVGTAVAWYQQKPGKAPKLLIYWASTRHTGVPSRFSGSGSGTDFTLTISSLQPEDFATYYCQQYSSYRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSEVQLVESGGGLVQPGGSLRLSCAASGFTFSSYVMSWVRQAPGKGLEWVATISSGGSYTYYPDSVKGRFTISRDNAKNTLYLQMNSLRAEDTAVYYCARRGDSMITTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 16 - protein, 710 aa, Artificial Sequence
  Lexatumumab-hFerr
LEELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 17 - protein, 709 aa, Artificial Sequence
  Lexatumumab-hFerr
LEELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 18 - protein, 708 aa, Artificial Sequence
  Drozitumab-hFerr
SELTQDPAVSVALGQTVRITCSGDSLRSYYASWYQQKPGQAPVLVIYGANNRPSGIPDRFSGSSSGNTASLTITGAQAEDEADYYCNSADSSGNHVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGGGVERPGGSLRLSCAASGFTFDDYAMSWVRQAPGKGLEWVSGINWQGGSTGYADSVKGRVTISRDNAKNSLYLQMNSLRAEDTAVYYCAKILGAGRGWYFDYWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 19 - protein, 707 aa, Artificial Sequence
  Drozitumab-hFerr
SELTQDPAVSVALGQTVRITCSGDSLRSYYASWYQQKPGQAPVLVIYGANNRPSGIPDRFSGSSSGNTASLTITGAQAEDEADYYCNSADSSGNHVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGGGVERPGGSLRLSCAASGFTFDDYAMSWVRQAPGKGLEWVSGINWQGGSTGYADSVKGRVTISRDNAKNSLYLQMNSLRAEDTAVYYCAKILGAGRGWYFDYWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 20 - protein, 708 aa, Artificial Sequence
  CM005G08-hFerr
ELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 21 - protein, 707 aa, Artificial Sequence
  CM005G08-hFerr
ELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 22 - protein, 714 aa, Artificial Sequence
  CM059H03-hFerr
ALETTLTQSPGTLSLSPGERATLSCRASQSISSSNLAWYQQKPGRAPRLLIYGASSRAIGIPDRFSGSGSGTDFTLTISRLEAEDFAVYYCQQYGSSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSEVQLVQSGAEVKKPGASVKVSCRASGYTFTSYGITWVRQAPGQGLEWMGWISAYNGKTNYVQELQGRVTMTTDTSTSTVYMELTSLRSDDTAVYYCARRGNNYRFGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 23 - protein, 713 aa, Artificial Sequence
  CM059H03-hFerr
ALETTLTQSPGTLSLSPGERATLSCRASQSISSSNLAWYQQKPGRAPRLLIYGASSRAIGIPDRFSGSGSGTDFTLTISRLEAEDFAVYYCQQYGSSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSEVQLVQSGAEVKKPGASVKVSCRASGYTFTSYGITWVRQAPGQGLEWMGWISAYNGKTNYVQELQGRVTMTTDTSTSTVYMELTSLRSDDTAVYYCARRGNNYRFGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 24 - protein, 713 aa, Artificial Sequence
  CM084A02-hFerr
AQSVLTQPPSASGTPGQRVSISCSGSSSNIGSNTVIWYQQLPGTAPKLLMYSNDRRPSGVPDRFSGSKSGTSASLAISGLQSEDEADYYCATWDDSLNGHYVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGAEVKKPGASVKLSCKASGYTLVNYFMHWVRQAPGQGPEWMGMINPSGGTTKNRQKFQDRVTMTRDTSTRTVYMELSGLTSEDTAVYYCATDFKGTDILFRDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 25 - protein, 712 aa, Artificial Sequence
  CM084A02-hFerr
AQSVLTQPPSASGTPGQRVSISCSGSSSNIGSNTVIWYQQLPGTAPKLLMYSNDRRPSGVPDRFSGSKSGTSASLAISGLQSEDEADYYCATWDDSLNGHYVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGAEVKKPGASVKLSCKASGYTLVNYFMHWVRQAPGQGPEWMGMINPSGGTTKNRQKFQDRVTMTRDTSTRTVYMELSGLTSEDTAVYYCATDFKGTDILFRDWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 26 - protein, 710 aa, Artificial Sequence
  T1014A04-hFerr
AQSVLTQPPSASGSPGQSVTISCTGTTSDVGGYNYVSWYQQHPGKAPKLMIYGVNQRPSGVPDRFSGSKSGNTASLTVSGLQAEDEADYYCSSYAGSNNWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGADVKRPGASVKVSCKISGDSFNAYFIHWVRQAPGQGLEWMGWFNPDSGTADSAQKFHGRVTMTRDTSSSTAFLELSRLRSDDTAVYYCVRQHRGNTFAPWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 27 - protein, 709 aa, Artificial Sequence
  T1014A04-hFerr
AQSVLTQPPSASGSPGQSVTISCTGTTSDVGGYNYVSWYQQHPGKAPKLMIYGVNQRPSGVPDRFSGSKSGNTASLTVSGLQAEDEADYYCSSYAGSNNWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGADVKRPGASVKVSCKISGDSFNAYFIHWVRQAPGQGLEWMGWFNPDSGTADSAQKFHGRVTMTRDTSSSTAFLELSRLRSDDTAVYYCVRQHRGNTFAPWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 28 - protein, 710 aa, Artificial Sequence
  T1014G03-hFerr
AQSALTQPASVSGSPGQSITISCTGTSSDIGAYKYVSWYQQHPGKAPKLVIYEVSNRPSGVSSRFSGSKSGQTASLTISGLQADDEADYYCNSYQGYNTWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGAEVKMPGASVKLSCRVSGDTFTAYFIHWVRQAPGQGLEWMGWFNPISGTAGSAEKFRGRVAMTRDTSISTAYMELNRLTFDDTAVYYCARQHRGNTFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 29 - protein, 710 aa, Artificial Sequence; identical to SEQ ID NO: 28
  T1014G03-hFerr

SEQ ID NO: 30 - protein, 709 aa, Artificial Sequence
  T1014A02-hFerr
ALSYVLTQPPSASGTPGQRVTISCAGSSSNIGGNTVNWYQQLPATAPKLLIYSNNQRPSGVPDRFSGSKSGTSASLAISGLQSEDEADYYCATWDDSRGGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSQVQLQESGPGLVKPSETLSLTCTVSGGSISDYYWSWVRQSPGKGLEWIGSIDYAGSTNYNPSLKSRVTMTIDKSKKQFPLKIDSVTAADTAMYYCARQLGRISDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 31 - protein, 708 aa, Artificial Sequence
  T1014A02-hFerr
ALSYVLTQPPSASGTPGQRVTISCAGSSSNIGGNTVNWYQQLPATAPKLLIYSNNQRPSGVPDRFSGSKSGTSASLAISGLQSEDEADYYCATWDDSRGGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSQVQLQESGPGLVKPSETLSLTCTVSGGSISDYYWSWVRQSPGKGLEWIGSIDYAGSTNYNPSLKSRVTMTIDKSKKQFPLKIDSVTAADTAMYYCARQLGRISDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 32 - protein, 710 aa, Artificial Sequence
  T1014A12-hFerr
AQSALTQPASVSGPPGQSITISCTGSSSDVGGYKYVSWYQQHPGKAPKLIIHDVSRRPSEVSSRFSGSKSGNTASLTISGLQAEDEAEYYCSSYSSTNSWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGADVKRPGASVKVSCKISGDSFTAYFIHWVRQAPGQGLEWMGWFNPDSGTADSAQKFHGRVTMTRDTSSSTAFLELSRLRSDDTAVYYCVRQHRGNTFAPWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 33 - protein, 709 aa, Artificial Sequence
  T1014A12-hFerr
AQSALTQPASVSGPPGQSITISCTGSSSDVGGYKYVSWYQQHPGKAPKLIIHDVSRRPSEVSSRFSGSKSGNTASLTISGLQAEDEAEYYCSSYSSTNSWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGADVKRPGASVKVSCKISGDSFTAYFIHWVRQAPGQGLEWMGWFNPDSGTADSAQKFHGRVTMTRDTSSSTAFLELSRLRSDDTAVYYCVRQHRGNTFAPWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 34 - protein, 710 aa, Artificial Sequence
  T1014B01-hFerr
AQSVVTQPPSVSGSPGQSVTISCTGTSSDIGAYNYVSWFQQHPGKAPKLIISEVSKRPSGVPDRLSGSKSGNTASLTVSGLQAEDEADYYCGSYAGSNIWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSQVQLVQSGAEVKKPGASVKVSCKISGDTFAAYFIHWVRQAPGQGLEWMGWFNPNSGTADSSQKFHGRVTMTRDTSISTAYMELSRLRSDDTAVYYCARQHRSNTFDPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 35 - protein, 709 aa, Artificial Sequence
  T1014B01-hFerr
AQSVVTQPPSVSGSPGQSVTISCTGTSSDIGAYNYVSWFQQHPGKAPKLIISEVSKRPSGVPDRLSGSKSGNTASLTVSGLQAEDEADYYCGSYAGSNIWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSQVQLVQSGAEVKKPGASVKVSCKISGDTFAAYFIHWVRQAPGQGLEWMGWFNPNSGTADSSQKFHGRVTMTRDTSISTAYMELSRLRSDDTAVYYCARQHRSNTFDPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 36 - protein, 710 aa, Artificial Sequence
  T1014Bll-hFerr
AQSALTQPASVSGSPGQSITISCTGTNSDVGGYNYVSWYQQHPGKAPKLMIYEVNNRPSGVSNRFSGSKSGNTASLTISGLQADDEADYYCSSYTTSNTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGAEVKKPGASVKVSCKISGDSFTAYFIHWLRQAPGEGLEWMGWFNPISGTAGSPQKFHGRVAMTRDTSISTAYMELTRLASDDTAIYYCARQHHSNTFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 37 - protein, 709 aa, Artificial Sequence
  T1014Bll-hFerr
AQSALTQPASVSGSPGQSITISCTGTNSDVGGYNYVSWYQQHPGKAPKLMIYEVNNRPSGVSNRFSGSKSGNTASLTISGLQADDEADYYCSSYTTSNTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGAEVKKPGASVKVSCKISGDSFTAYFIHWLRQAPGEGLEWMGWFNPISGTAGSPQKFHGRVAMTRDTSISTAYMELTRLASDDTAIYYCARQHHSNTFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 38 - protein, 710 aa, Artificial Sequence
  T1014F08-hFerr
ALPVLTQPPSASGSPGQSVTISCTGTSSDVGGYKYVSWYQQHPGKAPKLMIYEVSMRPSGVPDRFSGSKSGNTASLTVSGLQAEDEADYYCASYAGSNNWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGAEVKKPGASVKLSCRVSGDTFTAYFIHWVRQAPGQGPEWMGWFNPISGTAGSAARFRGRVAMTRDTSISTAYMELNRLTFDDTAVYYCARQHRGNTFDPWGKGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 39 - protein, 709 aa, Artificial Sequence
  T1014F08-hFerr
ALPVLTQPPSASGSPGQSVTISCTGTSSDVGGYKYVSWYQQHPGKAPKLMIYEVSMRPSGVPDRFSGSKSGNTASLTVSGLQAEDEADYYCASYAGSNNWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGAEVKKPGASVKLSCRVSGDTFTAYFIHWVRQAPGQGPEWMGWFNPISGTAGSAARFRGRVAMTRDTSISTAYMELNRLTFDDTAVYYCARQHRGNTFDPWGKGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 40 - protein, 710 aa, Artificial Sequence
  T1014G04-hFerr
AQPVLTQPPSASGSPGQSVTISCTGTSSDVGSYEYVSWYQQHPGKAPRLMISEVNKRPSGVPNRFSGSKSGNTASLTVSGLQADDEADYYCSSYAGSNNWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGADVKRPGASVKVSCKISGDSFTAYFIHWVRQAPGQGLEWMGWFNPDSGTADSAQKFHGRVTMTRDTSSSTAFLELSRLRSDDTAVYYCVRQHRGNTFAPWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 41 - protein, 709 aa, Artificial Sequence
  T1014G04-hFerr
AQPVLTQPPSASGSPGQSVTISCTGTSSDVGSYEYVSWYQQHPGKAPRLMISEVNKRPSGVPNRFSGSKSGNTASLTVSGLQADDEADYYCSSYAGSNNWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLVQSGADVKRPGASVKVSCKISGDSFTAYFIHWVRQAPGQGLEWMGWFNPDSGTADSAQKFHGRVTMTRDTSSSTAFLELSRLRSDDTAVYYCVRQHRGNTFAPWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 42 - protein, 715 aa, Artificial Sequence
  T1015A02-hFerr
AQAVLTQPSSASGTPGQRVTIPCSGSSSNIGGNTVNWYQQLPGTAPKLLIYGNDQRPSGVPDRFSGSKSGTSASLAITGLQSEDEADYYCAAWDDSLIGYVFGTGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSQVQLQESGPGLVKPSQTLSLKCNVSGGSIGTGDYYWSWIRQPPGKGLEWIGYIHSSGSTYYKPSLRSRLTVSMDTSRNQFSLKLTSVTAADTALYYCVREWANGDHWSAFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 43 - protein, 714 aa, Artificial Sequence
  T1015A02-hFerr
AQAVLTQPSSASGTPGQRVTIPCSGSSSNIGGNTVNWYQQLPGTAPKLLIYGNDQRPSGVPDRFSGSKSGTSASLAITGLQSEDEADYYCAAWDDSLIGYVFGTGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSQVQLQESGPGLVKPSQTLSLKCNVSGGSIGTGDYYWSWIRQPPGKGLEWIGYIHSSGSTYYKPSLRSRLTVSMDTSRNQFSLKLTSVTAADTALYYCVREWANGDHWSAFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 44 - protein, 710 aa, Artificial Sequence
  T1015A07 -hFerr
AQSALTQPASMSGSPGQSITISCTGTSSDVGGYNYVSWYQQHPGKAPKLMIYAVTNRPSGVSNRFSASKSGNTASLTISGLQAEDEADYYCSSYTSSNTWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLAQSGAEVNKPGASVKVSCKISGDSFTAYFIHWLRQAPGEGLEWMGWFNPISGTADSPQKFHGRVAMTRDTSISTAYMELTRLASDDTAIYYCARQHHSNTFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 45 - protein, 709 aa, Artificial Sequence
  T1015A07 -hFerr
AQSALTQPASMSGSPGQSITISCTGTSSDVGGYNYVSWYQQHPGKAPKLMIYAVTNRPSGVSNRFSASKSGNTASLTISGLQAEDEADYYCSSYTSSNTWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLAQSGAEVNKPGASVKVSCKISGDSFTAYFIHWLRQAPGEGLEWMGWFNPISGTADSPQKFHGRVAMTRDTSISTAYMELTRLASDDTAIYYCARQHHSNTFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 46 - protein, 714 aa, Artificial Sequence
  T1006F07-hFerr
AQSVLTQPPSVSVSPGQAARITCSGDKLGDKYASWYQQRPGQSPVLVIYQDNKRPSGIPERFSGSNSGNTATLKISGTQAMDEADYYCLAWDSSADWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAREPSFQQWGHYSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 47 - protein, 713 aa, Artificial Sequence
  T1006F07-hFerr
AQSVLTQPPSVSVSPGQAARITCSGDKLGDKYASWYQQRPGQSPVLVIYQDNKRPSGIPERFSGSNSGNTATLKISGTQAMDEADYYCLAWDSSADWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAREPSFQQWGHYSYGMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 48 - protein, 715 aa, Artificial Sequence
  42/43-hFerr
LEDIQMIQSPLSLPVIPGEPASMSCRSSRSLLHSNGNNYLQWYLQKPGQSPQLLIYLGSNRASGVPDRFSGSGSGTDFTLKISRVEAEDVGIYYCMQGLQLPTTFGGTKVIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLVQSGAEVKKPGASVKVSCKASGYTFTNYDINWVRQAPGQGLEWMGISAYTGNTNYAQKLQGRVTMTTDTSTSTAYMELRSLRSDDTAVYYCVRDYHDSNGYYYFDYWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 49 - protein, 714 aa, Artificial Sequence
  42/43-hFerr
LEDIQMIQSPLSLPVIPGEPASMSCRSSRSLLHSNGNNYLQWYLQKPGQSPQLLIYLGSNRASGVPDRFSGSGSGTDFTLKISRVEAEDVGIYYCMQGLQLPTTFGGTKVIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLVQSGAEVKKPGASVKVSCKASGYTFTNYDINWVRQAPGQGLEWMGISAYTGNTNYAQKLQGRVTMTTDTSTSTAYMELRSLRSDDTAVYYCVRDYHDSNGYYYFDYWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 50 - protein, 720 aa, Artificial Sequence
  44/45-hFerr
LEEIVLTQSPFFQSVTPKEKVTITCRASQNIGSSLHWYQQKPDQSPKLLIKSASQSFSGVPSRFSGSGSGTDFTLTINSLEAEDAATYYCHQSSSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLVQSGGGVVQPGRSLRLSCAASGFTFRTYGMHWVRQAPGKGLEWVAVLWYDGTNKYYADSVKGRFAISRDNSNNTLYLQMNSLRAEDAAVYYCARDGSYYYDSSGYYYVGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 51 - protein, 719 aa, Artificial Sequence
  44/45-hFerr
LEEIVLTQSPFFQSVTPKEKVTITCRASQNIGSSLHWYQQKPDQSPKLLIKSASQSFSGVPSRFSGSGSGTDFTLTINSLEAEDAATYYCHQSSSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLVQSGGGVVQPGRSLRLSCAASGFTFRTYGMHWVRQAPGKGLEWVAVLWYDGTNKYYADSVKGRFAISRDNSNNTLYLQMNSLRAEDAAVYYCARDGSYYYDSSGYYYVGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 52 - protein, 719 aa, Artificial Sequence
  46/47-hFerr
LEEVVLTQSPGTLSLSLGERATLSCRASQSVSSYLAWYQHKPGQAPRLLIYGTSSRATGIPDRFSGSGSGTNFTLTISRLEPEDFAVYYCQQYGSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLVESGGGVVQPGRSLRLSCSASGFTFSSGIHWVRQAPGKGLEWVVVMWYAGSNEYYADSVKGRFTISRDNSKNTLYLQMNSLRAEDTAVYYCARDQGVLLRFGELRGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 53 - protein, 718 aa, Artificial Sequence
  46/47-hFerr
LEEVVLTQSPGTLSLSLGERATLSCRASQSVSSYLAWYQHKPGQAPRLLIYGTSSRATGIPDRFSGSGSGTNFTLTISRLEPEDFAVYYCQQYGSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLVESGGGVVQPGRSLRLSCSASGFTFSSGIHWVRQAPGKGLEWVVVMWYAGSNEYYADSVKGRFTISRDNSKNTLYLQMNSLRAEDTAVYYCARDQGVLLRFGELRGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 54 - protein, 726 aa, Artificial Sequence
  Fc-hFerr LALAP I253A
DKTHTCPPCPAPEAAGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPEAAGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 55 - protein, 725 aa, Artificial Sequence
  Fc-hFerr LALAP I253A
DKTHTCPPCPAPEAAGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPEAAGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 56 - protein, 175 aa, Artificial Sequence; identical to SEQ ID NO: 11
  hFTL

SEQ ID NO: 57 - protein, 90 aa, Artificial Sequence; identical to SEQ ID NO: 1
  N_hFTL

SEQ ID NO: 58 - protein, 85 aa, Artificial Sequence; identical to SEQ ID NO: 2
  C_hFTL

SEQ ID NO: 59 - protein, 227 aa, Artificial Sequence; identical to SEQ ID NO: 8
  IgG1 Fc

SEQ ID NO: 60 - protein, 524 aa, Artificial Sequence
  IgG1 scFc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 215 aa, Artificial Sequence; identical to SEQ ID NO: 9
  Cona LC

SEQ ID NO: 62 - protein, 452 aa, Artificial Sequence
  Cona HC
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 510 aa, Artificial Sequence
  Cona scFab
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

(19) United States
(12) Patent Application Publication
(10) Pub. No.: US 2024/0182544 A1
(43) Pub. Date: Jun. 6, 2024

(54) POLYPEPTIDES TARGETING DR4 AND/OR DR5 AND RELATED COMPOSITIONS AND METHODS

(71) Applicant: The Hospital for Sick Children, Toronto (CA)

(72) Inventors: Jean-Philippe Julien, Toronto (CA); Edurne Rujas Diez, Vitoria (ES)

(21) Appl. No.: 18/253,483

(22) PCT Filed: Nov. 25, 2021
(86) PCT No.: PCT/CA2021/051690

§ 371 Date: May 18, 2023

Related U.S. Application Data
(60) Provisional application No. 63/118,306, filed on Nov. 25, 2020.

(51) Int. Cl. C07K 14/705; A61P 35/00; C07K 16/28

(52) U.S. Cl. CPC C07K 14/70578; A61P 35/00; C07K 16/2878; A61K 2039/505; C07K 2317/21; C07K 2317/24; C07K 2317/35; C07K 2317/52; C07K 2317/73; C07K 2319/30; C07K 2319/735

(57) ABSTRACT

A fusion protein comprises a nanocage monomer or a subunit thereof linked to a DR4 and/or DR5 antigen-binding moiety, wherein a plurality of the fusion proteins self-assemble to form a nanocage. Also described is a nanocage comprising the fusion protein and related compositions as well as methods and uses for treating and/or preventing cancer.

### CROSS-REFERENCE TO RELATED APPLICATIONS

This patent application is a U.S. National Stage Application of International Patent Application No. PCT/CA2021/051690, filed Nov. 25, 2021, which claims priority to and the benefit of U.S. Provisional Patent Application No. 63/118,306, filed Nov. 25, 2020, the contents of which are incorporated herein by reference in their entirety.

### SEQUENCE LISTING

The instant application contains a Sequence Listing which has been submitted electronically in ASCII format and is hereby incorporated by reference in its entirety. The ASCII copy, created on May 18, 2023, is named RBT-004WOUS_SL.txt and is 305,401 bytes in size.

### FIELD

The present invention relates to polypeptides. In particular, the present invention relates to DR4- and/or DR5-specific polypeptides and related constructs, compositions, and methods.

### BACKGROUND

Nanoparticles have contributed to advancements in various disciplines. Their use has the potential to confer targeted delivery and allows the engineering of ordered micro-arrays, slow release and caged micro-environments for catalytic processes.

For the fabrication of nanoparticles that contain sensitive and metastable proteins, protein self-assembly is an attractive method. Indeed, self-assembled nanoparticles form under physiological conditions through non-covalent interactions and reliably yield uniform and often symmetric nanocapsules or nanocages. Self-assembling protein nanoparticles possess three distinct surfaces that can all be tweaked to convey added functionalities: exterior, interior and inter-subunits surfaces.

Fusion proteins comprising self-assembling proteins have been described. For example, it is known to display antigens on the exterior surface of assembled nanocages for use as vaccines.

Death Receptors 4 and 5 (DR4/DR5) are members of the TNF-receptor superfamily. DR4 and DR5 become activated by trimerization upon ligand-binding. When activated, these receptors deliver an intracellular apoptosis signal to the cell. DR4/DR5 is up-regulated in various types of cancer cells. DR4 and DR5 present attractive targets for cancer therapy. However, efficacy of a candidate therapeutic based on targeting DR4 and/or DR5 may be limited by factors such as, e.g., insufficient ability to cross-link the receptors in the cell membrane and the need to balance potency with other characteristics of the candidate therapeutic.

A need exists for improved compositions and methods for targeting DR4/DR5.

### SUMMARY OF THE INVENTION

In accordance with an aspect, there is provided a fusion protein comprising a nanocage monomer or a subunit thereof linked to a DR4 and/or DR5 antigen-binding moiety, wherein a plurality of the fusion proteins self-assemble to form a nanocage.

In an aspect, the DR4 and/or DR5 antigen-binding moiety targets the DR4 and/or DR5 ectodomain.

In an aspect, the DR4 and/or DR5 antigen-binding moiety decorates the interior and/or exterior surface, preferably the exterior surface, of the assembled nanocage.

In an aspect, the DR4 and/or DR5 antigen-binding moiety comprises an antibody or fragment thereof.

In an aspect, the antibody or fragment thereof comprises a Fab fragment.

In an aspect, the antibody or fragment thereof comprises a scFab fragment, a scFv fragment, a sdAb fragment, a VHH domain or a combination thereof.

In an aspect, the antibody or fragment thereof comprises a heavy and/or light chain of a Fab fragment.

In an aspect, the fusion protein comprises a DR4 antigen-binding moiety.

In an aspect, the DR4 antigen-binding moiety comprises a DR4 antigen-binding moiety of CM005G08, CM059H03, CM084A02, T1014A04, T1014G03, T1014A02, T1014A12, T1014B01, T1014BII, T1014F08, T1014G04, T1015A02, T1015A07, T1006F07, 42/43, 44/45, and/or 46/47.

In an aspect, the fusion protein comprises a DR5 antigen-binding moiety.

In an aspect, the DR5 antigen-binding moiety comprises an antigen-binding moiety of Tigatuzumab, Lexatumumab, Drozitumab, and/or Conatumumab.

In an aspect, the DR5 antigen-binding moiety comprises the antigen-binding moiety of Conatumumab.

In an aspect, the DR4 and/or DR5 antigen-binding moiety is linked at the N- or C-terminus of the nanocage monomer or subunit thereof, or wherein there is a first DR4 and/or DR5 antigen-binding moiety linked at the N-terminus and a second DR4 and/or DR5 antigen-binding moiety linked at the C-terminus of the nanocage monomer or subunit thereoef, wherein the first and second DR4 and/or DR5 antigen-binding moieties are the same or different.

In an aspect, the fusion protein comprises a nanocage monomer and the DR4 and/or DR5 antigen-binding moiety is linked at the N-terminus of the nanocage monomer.

In an aspect, the fusion protein comprises a first nanocage monomer subunit linked to the DR4 and/or DR5 antigen-binding moiety; wherein the first nanocage monomer subunit is capable of self-assembling with a second nanocage monomer subunit to form the nanocage monomer.

In an aspect, the DR4 and/or DR5 antigen-binding moiety is linked at the N- or C-terminus of the first nanocage monomer subunit, or wherein there is a first DR4 and/or DR5 antigen-binding moiety linked at the N-terminus and a second DR4 and/or DR5 antigen-binding moiety linked at the C-terminus of the first nanocage monomer subunit, wherein the first and second DR4 and/or DR5 antigen-binding moieties are the same or different.

In an aspect, the fusion protein is in combination with the second nanocage monomer subunit.

In an aspect, the second nanocage monomer subunit is linked to a bioactive moiety at the N- or C-terminus.

In an aspect, the bioactive moiety comprises an Fc fragment.

In an aspect, the Fc fragment is an IgG1 Fc fragment.

In an aspect, the Fc fragment comprises one or more mutations or sets of mutations that modulate the half-life of the fusion protein from, for example, minutes or hours to several days, weeks, or months.

In an aspect, the Fc fragment comprises a mutation at one or more of L234, L235, G236, G237, M252, I253, S254, T256, P329, A330, M428, N434, or a combination thereof (wherein numbering is according to the EU index), such as M428L and N434S (“LS”); M252Y, S254T and T256E (“YTE”); L234A and L235A (“LALA”); I253A, and/or L234A, L235A, and P329G (“LALAP”), G236R, G237A, A330L or a combination thereof.

In an aspect, the Fc fragment is an scFc fragment.

In an aspect, from about 3 to about 100 nanocage monomers, such as 24, 32, 48, or 60 monomers, or from about 4 to about 200 nanocage monomer subunits, such as 4, 6, 8, 10, 12, 14, 18, 20, 22, 24, 26, 28, 30, 32, 34, 36, 38, 40, 42, 44, 46, 48, 50, or more, optionally in combination with one or more whole nanocage monomers, are capable of self-assembling to form a nanocage.

In an aspect, the nanocage monomer is selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof.

In an aspect, the nanocage monomer is apoferritin, optionally human apoferritin.

In an aspect, the nanocage monomer is an apoferritin light chain, optionally human apoferritin light chain.

In an aspect, the fusion protein comprises a first apoferritin subunit, optionally a first human apoferritin subunit, and wherein the first apoferritin subunit is capable of self-assembling with a second apoferritin subunit.

In an aspect, the first and second apoferritin monomer subunits interchangeably comprise the “N” and “C” regions of apoferritin.

In an aspect, the “N” region of apoferritin comprises or consists of a sequence at least 70% (such as at least 75%, 80%, 85%, 90%, 95%, 96%, 97%, 98%, 99%, or 100%) identical to:

(SEQ ID NO: 1)
MSSQIRQNYSTDVEAAVNSLVNLYLQASYTYLSLGFYFDRDDVALEGVS
HFFRELAEEKREGYERLLKMQNQRGGRALFQDIKKPAEDEW.

In an aspect, the “C” region of apoferritin comprises or consists of a sequence at least 70% (such as at least 75%, 80%, 85%, 90%, 95%, 96%, 97%, 98%, 99%, or 100%) identical to:

(SEQ ID NO: 2)
GKTPDAMKAAMALEKKLNQALLDLHALGSARTDPHLCDFLETHFLDEEV
KLIKKMGDHLTNLHRLGGPEAGLGEYLFERLTLRHD;
or
(SEQ ID NO: 3)
GKTPDAMKAAMALEKKLNQALLDLHALGSARTDPHLCDFLETHFLDEEV
KLIKKMGDHLTNLHRLGGPEAGLGEYLFERLTLKHD.

In an aspect, the fusion protein further comprises a bioactive moiety and a linker between the nanocage monomer or subunit thereof and the bioactive moiety.

In an aspect, the linker is flexible or rigid and comprises from about 1 to about 30 amino acid residues, such as from about 8 to about 16 amino acid residues.

In an aspect, the linker comprises a GGGGS repeat, such as 1, 2, 3, 4, or more GGGGS repeats.

In an aspect, the linker comprises or consists of a sequence at least 70% (such as at least 75%, 80%, 85%, 90%, 95%, 96%, 97%, 98%, 99%, or 100%) identical to:

(SEQ ID NO: 4)
GGGGSGGGGSGGGGSGGGGSGGGGSGG.

In an aspect, the fusion protein further comprises a C-terminal linker.

In an aspect, the C-terminal linker comprises a GGS repeat.

In an aspect, the C-terminal linker comprises or consists of a sequence at least 70% (such as at least 75%, 80%, 85%, 90%, 95%, 96%, 97%, 98%, 99%, or 100%) identical to:

(SEQ ID NO: 5)
GGSGGSGGSGGSGGGSGGSGGSGGSG.

In accordance with an aspect, there is provided a nanocage comprising at least one fusion protein described herein and at least one second nanocage monomer or subunit thereof that self-assembles with the fusion protein.

In an aspect, the fusion protein comprises a first nanocage monomer subunit, the second nanocage monomer or subunit thereof is a second nanocage monomer subunit, and the second nanocage monomer subunit self-assembles with the fusion protein to form the nanocage monomer.

In an aspect, each nanocage monomer comprises the fusion protein described herein.

In an aspect, from about 1% to about 100%, such as from about 1%, 4%, 8%, 10%, 12%, 15%, 20%, 25%, 30%, 35%, 40%, 45%, 50%, 55%, 60%, 65%, 70%, 75%, 80%, 85%, 90%, or 95%, to about 4%, 8%, 10%, 12%, 15%, 20%, 25%, 30%, 35%, 40%, 45%, 50%, 55%, 60%, 65%, 70%, 75%, 80%, 85%, 90%, 95%, or 100%, such as from about 20% to about 80%, of the nanocage monomers or subunits thereof comprise the fusion protein described herein.

In an aspect, the nanocage comprises at least 2, 3, 4, 5, 6, 7, 8, 9, or 10 different DR4 and/or DR5 antigen-binding moieties, such as 2 or 3 different DR4 and/or DR5 antigen-binding moieties.

In an aspect, the nanocage is multivalent.

In an aspect, the nanocage is multispecific.

In an aspect, at least one DR4 and/or DR5 antigen-binding moiety decorates the exterior surface of the nanocage and at least one Fc fragment decorates the exterior surface of the nanocage.

In an aspect, at least two DR4 and/or DR5 antigen-binding moieties decorate the exterior surface of the nanocage and at least two Fc fragments decorate the exterior surface of the nanocage.

In an aspect, the nanocage comprises a 4:1:1 ratio of an antigen-binding moiety, such as an Fab fragment, of Conatumumab fused to a first full length human ferritin light chain; an Fc fragment (optionally an scFc fragment) fused to a second full length human ferritin light chain; and a third human ferritin light chain.

In an aspect, the nanocage comprises at least one DR4 and/or DR5 antigen-binding moiety fused to the N-terminus of a full ferritin monomer, at least one DR4 and/or DR5 antigen-binding moiety fused to the N-terminus of an N-ferritin monomer subunit, and an Fc fragment fused to the N-terminus of a C-ferritin monomer subunit.

In an aspect, the nanocage comprises a 2:1:1 ratio of the DR4 and/or DR5 antigen-binding moiety fused to the N-terminus of the full ferritin monomer: the DR4 and/or DR5 antigen-binding moiety fused to the N-terminus of the N-ferritin monomer subunit: the Fc fragment fused to the N-terminus of the C-ferritin monomer subunit.

In an aspect, the nanocage comprises at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 DR4 and/or DR5 antigen-binding moieties.

In an aspect, the nanocage comprises at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 bioactive moieties.

In an aspect, the nanocage is carrying a cargo molecule, such as a pharmaceutical agent, a diagnostic agent, and/or an imaging agent.

In an aspect, the cargo molecule is not fused to the fusion protein and is contained in the nanocage internally.

In an aspect, the cargo molecule is a protein and is fused to the fusion protein such that the cargo molecule is contained in the nanocage internally.

In an aspect, the cargo molecule comprises a fluorescent protein, such as GFP, EGFP, Ametrine, and/or a flavin-based fluorescent protein, such as a LOV-protein, such as iLOV.

In an aspect, the nanocage is capable of killing DR4- and/or DR-5-positive cancer cells with an IC₅₀ value of less than about 0.1 μg/ml, less than about 0.01 μg/ml, or less than about 0.001 μg/ml, as determined in an in vitro cell killing assay.

In an aspect, the nanocage is capable of killing DR4- and/or DR-5-positive cancer cells with an IC₅₀ value of less than about 10 pM, less than about 1 pM, or less than about 0.1 pM, as determined in an in vitro cell killing assay.

In an aspect, the nanocage is capable of killing DR4- and/or DR-5-positive cancer cells with an IC₅₀ value that is at least about 10, at least about 100, at least about 1000, at least about 10,000, or at least about 100,000 more potent than the corresponding IgG on a mass and/or molar basis.

In accordance with an aspect, there is provided a DR4 and/or DR5 therapeutic or prophylactic composition comprising the nanocage described herein.

In accordance with an aspect, there is provided a nucleic acid molecule encoding the fusion protein described herein.

In accordance with an aspect, there is provided a vector comprising the nucleic acid molecule described herein.

In accordance with an aspect, there is provided a host cell comprising the vector described herein and producing the fusion protein described herein.

In accordance with an aspect, there is provided a method for treating and/or preventing cancer, the method comprising administering the nanocage or the composition described herein.

In an aspect, the cancer is selected from the group consisting of breast cancer, colon cancer, lymphoma, or lung cancer.

In accordance with an aspect, there is provided a use of the nanocage or the composition described herein for treating and/or preventing cancer.

In an aspect, the nanocage or the composition described herein is for use in treating and/or preventing cancer.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1. Multabody assembly displays Fabs to target trimeric receptors. Schematic representation showing Multabody valency (right) in comparison to a conventional IgG (left). Close-up view of Fab (dark red for the heavy chain and light red for light chain) clustering at the three-fold symmetry axes (light teal) of ferritin. Fragments in gold represent Fc fragments.

FIG. 2. Avidity enhances cell death against multiple cancer cell lines. Relative killing capacity of multiple cancer cell lines by Tigatuzumab in the Multabody format vs the parental IgG.

FIG. 3. Avidity enhances cell death against multiple cancer cell lines. Relative killing capacity of multiple cancer cell lines by Conatumumab in the Multabody format vs the parental IgG.

FIG. 4A is a diagrammatic representation of human ferritin light chain (hFTL) and exemplary N-half ferritin (N-hFTL) and C-half ferritin (C-hFTL) molecules.

FIG. 4B is a diagrammatic representation of fusion polypeptides that together form exemplary Multabodies of the disclosure.

FIG. 5 is a graph showing tumor volumes on Day 88 in mice bearing colon cancer xenograft tumors and treated with vehicle, DR5 IgG, or DR5 MB. Statistical analyses were performed using a Mann-Whitney test.

### DETAILED DESCRIPTION OF CERTAIN ASPECTS

Upon ligand-binding, Death Receptors 4 and 5 (DR4 and DR5) may trimerize and deliver an intracellular apoptosis signal to a cell (e.g., cancer cell). The present disclosure encompasses the recognition that increased valency of a molecule targeting DR4 and/or DR5 may correspond to increased potency of a candidate therapeutic.

Described herein are systems for targeting DR4 and/or DR5 using nanocages that comprise multiple DR4 and/or DR5 antigen-binding moieties, such as Fab fragments capable of binding DR4 and/or DR5. In aspects, additional bioactive moieties such as Fc fragments are also displayed by the nanocages. Also described are methods of treatment (e.g., of cancer) using such nanocages. Systems disclosed herein use a “Multabody” platform that allows modulation of binding and pharmacokinetic features of the nanocages, e.g., by controlling the number or ratio of Fab and Fc molecules within nanocages. The nanocages can contain as many DR4 and/or DR5 antigen-binding moieties and/or bioactive moieties as there are monomers in the nanocage, for example in the case of ferritin, 24. In aspects, some or all of the monomers are split so that up to double the number of DR4 and/or DR5 antigen-binding moieties and/or bioactive moieties can decorate the nanocage, for example in the case of ferritin, 48. Features of the presently disclosed system for targeting DR4 and/or DR5 can be fine-tuned, e.g., to balance potency and other considerations, such as toxicity to non-cancer cells.

Here, it is demonstrated that multimerization of DR4 and/or DR5-targeting Fabs on the Multabody platform, through its unique 3-fold symmetry axes, results in the greater ability to cross-link the cell surface receptors DR4 and/or DR5. As a consequence, the Multabody significantly enhances cellular signaling for killing cancer cells in comparison to the corresponding IgG. In aspects, the increased potency of the Multabodies is not necessarily increased at a 1:1 ratio, where a doubling in valency results in a doubling in potency. Instead, the potency is, in aspects, synergistically increased by at least 10-fold and in aspects much more. The therapeutic potential of this engineered molecule was demonstrated using various cancer cell lines.

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only, and is not intended to be limiting. Many patent applications, patents, and publications are referred to herein to assist in understanding the aspects described. Each of these references are incorporated herein by reference in their entirety.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically less than 0.1% by weight of non-specified component(s).

It will be understood that any component defined herein as being included may be explicitly excluded from the claimed invention by way of proviso or negative limitation. For example, in some aspects the nanocages and/or fusion proteins described herein may exclude a ferritin heavy chain and/or may exclude an iron-binding component.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The abbreviation, “e.g.” is derived from the Latin exempli gratia, and is used herein to indicate a non-limiting example. Thus, the abbreviation “e.g.” is synonymous with the terms “for example,” or “such as.” The word “or” is intended to include “and” unless the context clearly indicates otherwise.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

The terms “protein nanoparticle,” “nanocage,” and “Multabody” are used interchangeably herein and refer to a protein-based polyhedron shaped structure made from a plurality of nanocage monomers. These nanocage monomers, or subunits thereof, are each composed of proteins or polypeptides (for example a glycosylated polypeptide), and, optionally of single or multiple features of the following: nucleic acids, prosthetic groups, organic and inorganic compounds. Non-limiting examples of protein nanoparticles include ferritin nanoparticles (see, e.g., Zhang, Y. Int. J. Mol. Sci., 12:5406-5421, 2011, incorporated by reference herein), encapsulin nanoparticles (see, e.g., Sutter et al., Nature Struct, and Mol. Biol., 15:939-947, 2008, incorporated by reference herein), Sulfur Oxygenase Reductase (SOR) nanoparticles (see, e.g., Urich et al., Science, 311 :996-1000, 2006, incorporated by reference herein), lumazine synthase nanoparticles (see, e.g., Zhang et al., J. Mol. Biol., 306: 1099-1114, 2001) or pyruvate dehydrogenase nanoparticles (see, e.g., Izard et al., PNAS 96: 1240-1245, 1999, incorporated by reference herein). Ferritin, apoferritin, encapsulin, SOR, lumazine synthase, and pyruvate dehydrogenase are monomeric proteins that self-assemble into a globular protein complexes that in some cases consists of 24, 60, 24, 60, and 60 protein subunits, respectively. Ferritin and apoferritin are generally referred to interchangeably herein and are understood to both be suitable for use in the fusion proteins, nanocages, and methods described herein. Carboxysome, vault proteins, GroEL, heat shock protein, E2P and MS2 coat protein also produce nanocages are contemplated for use herein. In addition, fully or partially synthetic self-assembling monomers are also contemplated for use herein.

It will be understood that each nanocage monomer may be divided into two or more subunits that will self-assemble into a functional nanocage monomer. For example, ferritin or apoferritin may be divided into an N- and C- subunit, e.g., an N- and C- subunit obtained by dividing full-length ferritin substantially in half, so that each subunit may be separately bound to a different DR4 and/or DR5 antigen-binding moiety or bioactive moiety (e.g. Fc fragment) for subsequent self-assembly into a nanocage monomer and a nanocage. Each subunit may, in aspects, bind a DR4 and/or DR5 antigen-binding moiety and/or bioactive moiety at both termini, either the same or different. By “functional nanocage monomer or subunit thereof” it is intended that the nanocage monomer or subunit thereof is capable of self-assembly with complementary monomers or subunits into a nanocage as described herein.

The terms “ferritin” and “apoferritin” are used interchangeably herein and generally refer to a polypeptide (e.g., a ferritin chain) that is capable of assembling into a ferritin complex which typically comprises 24 protein subunits. It will be understood that the ferritin can be from any species. Typically, the ferritin is a human ferritin. In some embodiments, the ferritin is a wild-type ferritin. For example, the ferritin may be a wild-type human ferritin. In some embodiments, a ferritin light chain is used as a nanocage monomer, and/or a subunit of a ferritin light chain is used as a nanocage monomer subunit. In some embodiments, assembled nanocages do not include any ferritin heavy chains or other ferritin components capable of binding to iron.

The term “multispecific,” as used herein, refers to the characteristic of having at least two binding sites at which at least two different binding partners, e.g., an antigen or receptor (e.g., Fc receptor), can bind. For example, a nanocage that comprises at least two Fab fragments, wherein each of the two Fab fragments binds to a different antigen, is “multispecific.” As an additional example, a nanocage that comprises an Fc fragment (which is capable of binding to an Fc receptor) and an Fab fragment (which is capable of binding to an antigen) is “multispecific.”

The term “multivalent,” as used herein, refers to the characteristic of having at least two binding sites at which a binding partner, e.g., an antigen or receptor (e.g., Fc receptor), can bind. The binding partners that can bind to the at least two binding sites may be the same or different.

The term “antibody”, also referred to in the art as “immunoglobulin” (Ig), used herein refers to a protein constructed from paired heavy and light polypeptide chains; various Ig isotypes exist, including IgA, IgD, IgE, IgG, such as IgG₁, IgG₂, IgG₃, and IgG₄, and IgM. It will be understood that the antibody may be from any species, including human, mouse, rat, monkey, llama, or shark. When an antibody is correctly folded, each chain folds into a number of distinct globular domains joined by more linear polypeptide sequences. For example, in the case of IgGs, the immunoglobulin light chain folds into a variable (V_{L}) and a constant (CL) domain, while the heavy chain folds into a variable (V_{H}) and three constant (C_{H}, C_{H2}, C_{H3}) domains. Interaction of the heavy and light chain variable domains (V_{H} and V_{L}) results in the formation of an antigen binding region (Fv). Each domain has a well-established structure familiar to those of skill in the art.

The light and heavy chain variable regions are responsible for binding the target antigen and can therefore show significant sequence diversity between antibodies. The constant regions show less sequence diversity, and are responsible for binding a number of natural proteins to elicit important immunological events. The variable region of an antibody contains the antigen binding determinants of the molecule, and thus determines the specificity of an antibody for its target antigen. The majority of sequence variability occurs in six hypervariable regions, three each per variable heavy and light chain; the hypervariable regions combine to form the antigen-binding site, and contribute to binding and recognition of an antigenic determinant. The specificity and affinity of an antibody for its antigen is determined by the structure of the hypervariable regions, as well as their size, shape and chemistry of the surface they present to the antigen.

An “antibody fragment” as referred to herein may include any suitable antigen-binding antibody fragment known in the art. The antibody fragment may be a naturally-occurring antibody fragment, or may be obtained by manipulation of a naturally-occurring antibody or by using recombinant methods. For example, an antibody fragment may include, but is not limited to a Fv, single-chain Fv (scFv; a molecule consisting of V_{L} and V_{H} connected with a peptide linker), Fc, single-chain Fc, Fab, single-chain Fab, F(ab′)₂, single domain antibody (sdAb; a fragment composed of a single V_{L} or V_{H}), and multivalent presentations of any of these. As used herein, “antigen-binding moiety” refers to an antibody or portion of an antibody that specifically binds to a target antigen.

By the term “synthetic antibody” as used herein, is meant an antibody which is generated using recombinant DNA technology. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “epitope” refers to an antigenic determinant. An epitope is the particular chemical groups or peptide sequences on a molecule that are antigenic, that is, that elicit a specific immune response. An antibody specifically binds a particular antigenic epitope, e.g., on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, about 11, or about 8 to about 12 amino acids in a unique spatial conformation. Methods of determining spatial conformation of epitopes include, for example, x-ray crystallography and 2-dimensional nuclear magnetic resonance. See, e.g., “Epitope Mapping Protocols” in Methods in Molecular Biology, Vol. 66, Glenn E. Morris, Ed (1996).

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the aspects described herein include, but are not limited to, the use of partial nucleotide sequences of more than one gene and that these nucleotide sequences could be arranged in various combinations to elicit the desired immune response. Moreover, a skilled artisan will understand that an antigen need not be encoded by a “gene” at all. It is readily apparent that an antigen can be synthesized or can be derived from a biological sample. Such a biological sample can include, but is not limited to a tissue sample, a cell, or a biological fluid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, typically, a human.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of composition includes, e.g., subcutaneous (s.c.), intravenous (i.v.), intramuscular (i.m.), or intrasternal injection, or infusion techniques. Also included are inhalation and intranasal administration.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, “to treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment. As used herein, the terms “prevention” or “prophylaxis” refers to the reduction in the risk of acquiring or developing a disease or disorder, for example cancer, or the reduction or inhibition of the recurrence of a disease or disorder, for example cancer. Thus, a DR4- and/or DR-5 therapeutic or prophylactic composition refers to a composition comprising assembled nanocages as described herein, or fusion proteins as described herein that are capable of assembling into nanocages, that when administered to a subject are capable of treating and/or preventing a disease and/or condition in which DR4 and/or DR5 is implicated, such as cancer.

The terms “therapeutically effective amount”, “effective amount” or “sufficient amount” mean a quantity sufficient, when administered to a subject, including a mammal, for example a human, to achieve a desired result, for example an amount effective to cause a cell death (e.g., cancer cell death). Effective amounts of the compounds described herein may vary according to factors such as the molecule, age, sex, species, and weight of the subject. Dosage or treatment regimes may be adjusted to provide the optimum therapeutic response, as is understood by a skilled person. For example, administration of a therapeutically effective amount of the fusion proteins described herein is, in aspects, sufficient to treat and/or prevent cancer. Moreover, a treatment regime of a subject with a therapeutically effective amount may consist of a single administration, or alternatively comprise a series of applications. The frequency and length of the treatment period depends on a variety of factors, such as the molecule, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The fusion proteins described herein may, in aspects, be administered before, during or after treatment with conventional therapies for the disease or disorder in question. For example, the fusion proteins described herein may find particular use in combination with conventional treatments for cancer.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

“Variants” are biologically active fusion proteins, antibodies, or fragments thereof having an amino acid sequence that differs from a comparator sequence by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments of at least 10 amino acids that retain some level of the biological activity of the comparator sequence. Variants also include polypeptides wherein one or more amino acid residues are added at the N- or C-terminus of, or within, the comparative sequence. Variants also include polypeptides where a number of amino acid residues are deleted and/or optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

“Active” or “activity” for the purposes herein refers to a biological and/or an immunological activity of the fusion proteins described herein, wherein “biological” activity refers to a biological function (either inhibitory or stimulatory) caused by the fusion proteins.

The fusion proteins described herein may include modifications. Such modifications include, but are not limited to, conjugation to an effector molecule. Modifications further include, but are not limited to conjugation to detectable reporter moieties. Modifications that extend half-life (e.g., pegylation) are also included. Modifications for de-immunization are also included. Proteins and non-protein agents may be conjugated to the fusion proteins by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990), which is incorporated by reference herein and those described by Amon et al., Adv. Exp. Med. Biol. 303, 79-90 (1991) and by Kiseleva et al, Mol. Biol. (USSR)25, 508-514 (1991), both of which are incorporated by reference herein.

### Fusion Proteins

Described herein are fusion proteins. The fusion proteins comprise a nanocage monomer or subunit thereof linked to a DR4 and/or DR5 antigen-binding moiety. A plurality of the fusion proteins may self-assemble to form a nanocage. In this way, the DR4 and/or DR5 antigen-binding moiety may decorate the interior surface of the assembled nanocage, the exterior surface of the assembled nanocage, or both. In some embodiments, the DR4 and/or DR5 antigen-binding moiety decorates the exterior surface of the assembled nanocage.

The DR4 and/or DR5 antigen-binding moiety is typically an antibody or a fragment thereof that binds to DR4 and/or DR5. While the DR4 and/or DR5 antigen-binding moiety can target any part of the DR4 and/or DR5 receptor, it typically targets the ectodomain.

It will be understood that the antibody or fragment thereof may comprise or consist of, for example, a heavy and/or light chain of a Fab fragment. The antibody or fragment thereof may comprise or consist of a scFab fragment, a scFv fragment, a sdAb fragment, a nanobody and/or a VHH region for example. It will be understood that any antibody or fragment thereof may be used in the fusion proteins described herein.

Generally, the fusion protein described herein is associated with a Fab light chain and/or heavy chain, which may be produced separately or contiguously with the fusion protein.

In some aspects, the fusion protein comprises a DR4 antigen-binding moiety. The DR4 antigen-binding moiety may comprise, for example, an antigen-binding moiety of CM005G08, CM059H03, CM084A02, T1014A04, T1014G03, T1014A02, T1014A12, T1014B01, T1014BII, T1014F08, T1014G04, T1015A02, T1015A07, T1006F07, 42/43, 44/45, and/or 46/47.

In some aspects, the fusion protein comprises a DR5 antigen-binding moiety. The DR5 antigen-binding moiety may comprise, for example, an antigen-binding moiety of Tigatuzumab, Conatumumab, Drozitumab, and/or Lexatumumab. In some cases, these antigen-binding moieties are referred to herein interchangeably as, for example, Conatumumab, Cona, or Conatumumab IgG.

In certain aspects, the nanocage monomer described herein comprises a first nanocage monomer subunit linked to the DR4 and/or DR5 antigen-binding moiety or to a bioactive motive (e.g. Fc fragment). The first nanocage monomer subunit is capable of self-assembling with a second nanocage monomer subunit to form a full nanocage monomer, a plurality of which self-assemble to form the nanocage, thus allowing for multiple DR4 and/or DR5 antigen-binding moieties or other moieties to self assemble into one nanocage. Amounts of each different component are controlled by controlling gene and expression ratios. These nanocage monomer subunits can be used alone or in combination.

For example, the DR4 and/or DR5 antigen-binding moiety or the bioactive moiety (e.g. the Fc fragment) can be linked to a divided apoferritin monomer (N- or C-subunit, which are each typically about half of a full-length apoferritin monomer). Each subunit fused to the DR4 and/or DR5 antigen-binding moiety or the bioactive moiety (e.g. Fc fragment) self-assembles into an apoferritin monomer that in turn self-assembles with other apoferritin monomers (either a full apoferritin or an assembled apoferritin formed of N- and C-subunits) to form a nanocage.

In aspects, the DR4 and/or DR5 antigen-binding moiety or the bioactive moiety (e.g. Fc fragment) is linked at the N- or C-terminus of the nanocage monomer or subunit thereof. In aspects, there is a first DR4 and/or DR5 antigen-binding moiety linked at the N-terminus and a second DR4 and/or DR5 antigen-binding moiety linked at the C-terminus of the nanocage monomer or subunit thereof, wherein the first and second DR4 and/or DR5 antigen-binding moieties are the same or different.

For example, in aspects, the fusion protein comprises a nanocage monomer and the DR4 and/or DR5 antigen-binding moiety is linked at the N-terminus of the nanocage monomer. In other aspects, the fusion protein comprises a first nanocage monomer subunit linked to the DR4 and/or DR5 antigen-binding moiety; wherein the first nanocage monomer subunit is capable of self-assembling with a second nanocage monomer subunit to form the nanocage monomer. In other aspects, the DR4 and/or DR5 antigen-binding moiety is linked at the N- or C-terminus of the first nanocage monomer subunit, or there is a first DR4 and/or DR5 antigen-binding moiety linked at the N-terminus and a second DR4 and/or DR5 antigen-binding moiety linked at the C-terminus of the first nanocage monomer subunit. As will be understood, the first and second DR4 and/or DR5 antigen-binding moieties may be the same or different.

The first nanocage monomer subunit described herein is, in aspects, provided in combination with the second nanocage monomer subunit, with which the first nanocage monomer subunit is capable of self-assembling. The second nanocage monomer subunit may or may not be a fusion protein. In some aspects, the second nanocage monomer subunit is linked to a bioactive moiety, such as an Fc fragment, optionally wherein the Fc fragment is an IgG1 Fc.

For example, in some aspects, the fusion protein comprises a first nanocage monomer subunit linked to the DR4 and/or DR5 antigen-binding moiety. In use, the first nanocage monomer subunit self-assembles with a second nanocage monomer subunit to form the nanocage monomer. As described above, a plurality of the nanocage monomers self-assemble to form a nanocage. The nanocage monomer subunits may be provided alone or in combination and may have the same or a different DR4 and/or DR5 antigen-binding moiety fused thereto or another bioactive moiety as described herein.

A nanocage made from the nanocage monomers and/or nanocage monomer subunits described herein may have bioactive moieties included in addition to one or more DR4 and/or DR5 antigen-binding moieties. The bioactive moiety may be any moiety capable of being a part of a fusion protein and is, typically a protein. Typically, the bioactive moiety comprises an antibody or fragment thereof, an antigen, a detectable moiety, a pharmaceutical agent, a diagnostic agent, or combinations thereof.

For example, the bioactive moiety may comprise, one or both chains of an Fc fragment. In the case of making a fusion protein that contains only one chain of the Fc fragment, nanocage self-assembly will typically lead to the assembly of the functional Fc fragment. In the case of fusing both chains of the Fc fragment, both chains will typically be linked through a flexible linker to allow folding of the Fc fragment.

The Fc fragment may be derived from any type of antibody as will be understood but is, typically, an IgG1 Fc fragment. The Fc fragment may further comprise one or more mutations that modulate the half-life and/or effector functions of the fusion protein and/or the resulting assembled nanocage comprising the fusion protein. For example, the Fc fragment may have a mutation at one or more of L234, L235, G236, G237, M252, I253, S254, T256, P329, A330, M428, N434, or a combination thereof (wherein numbering is according to the EU index). For example, in some embodiments, the Fc fragment comprises a L234A, L235A, M252Y, I253A, S254T, T256E, P329G, M428L, or N434S mutation, or a combination thereof. In some embodiments, the Fc fragment comprises sets of mutations such as: M428L and N434S (“LS”); M252Y, S254T and T256E (“YTE”); L234A and L235A (“LALA”); I253A, and/or L234A, L235A, and P329G (“LALAP”), G236R, G237A, A330L or a combination thereof. For example, the half-life of an Fc fragment comprising one or more mutations as described herein may be in the scale of minutes, days, weeks, or even months.

Moreover, other substitutions in the fusion proteins and nanocages described herein are contemplated, including Fc sequence modifications and addition of other agents (e.g. human serum albumin, human serum albumin peptide sequences and antibodies such as Fabs and/or nanobodies targeting human serum albumin), that allow changes in bioavailability and will be understood by a skilled person. Furthermore, the fusion proteins and nanocages described herein can be modulated in sequence or by addition of other agents to mute immunogenicity and anti-drug responses (therapeutic, e.g. matching sequence to host, or addition of immunosuppressive therapies [such as, for example, methotrexate when administering infliximab for treating rheumatoid arthritis or induction of neonatal tolerance, which is a primary strategy in reducing the incidence of inhibitors against FVIII (reviewed in: DiMichele D M, Hoots W K, Pipe S W, Rivard G E, Santagostino E. International workshop on immune tolerance induction: consensus recommendations. Haemophilia. 2007;13:1-22, incorporated herein by reference in its entirety]).

In cases where the antibody or fragment thereof comprises two chains, such as a first and second chain in the case of a Fc fragment, or a heavy and light chain, the two chains are optionally separated by a linker. The linker may be flexible or rigid, but it typically is flexible to allow the chains to fold appropriately. The linker is generally long enough to impart some flexibility to the fusion protein, although it will be understood that linker length will vary depending upon the nanocage monomer or subunit thereof and bioactive moiety sequences and the three-dimensional conformation of the fusion protein. Thus, the linker is typically from about 1 to about 130 amino acid residues, such as from about 1, 5, 10, 15, 20, 25, 30, 35, 40, 45, 50, 55, 60, 65, 70, 75, 80, 85, 90, 95, 100, 105, 110, 115, 120, or 125 to about 5, 10, 15, 20, 25, 30, 35, 40, 45, 50, 55, 60, 65, 70, 75, 80, 85, 90, 95, 100, 105, 110, 115, 120, 125, or 130 amino acid residues, such as from about 50 to about 90 amino acid residues, such as 70 amino acid residues.

The linker may be of any amino acid sequence and, in one typical example, the linker comprises a series of G and S amino acids, such as a series of GS repeats, GGS repeats, GGGS repeats, and/or GGGGS repeats. Typically, the linker comprises a GGGGS and/or GGGS repeat and, more typically, the linker comprises at least about 2, 3, 4, 5, 6, 7, 8, 9, 10, 11, 12, 13, 14, 15, 16, 17, 18, 19, or 20 GGGS and/or GGGGS repeats, such as about 5 GGGS repeats and/or about 14 GGGGS repeats. In specific aspects, the linker comprises or consists of a sequence at least 70% (such as at least 75%, 80%, 85%, 90%, 95%, 96%, 97%, 98%, 99%, or 100%) identical to:

(SEQ ID NO: 6)
GGGGSGGGGSGGGGSGGGGSGGGGSGGGGSGGGGGGGGSGGGGSGGGG
SGGGGSGGGGSGGGGSGGGGS.

In typical aspects, the antibody or fragment thereof binds specifically to an antigen associated with DR4 and/or DR5. Typically, the antigen is associated with the ectodomain of DR4 and/or DR5.

In a specific example, the antibody or fragment thereof comprises or consists of a sequence at least 70% (such as at least 75%, 80%, 85%, 90%, 95%, 96%, 97%, 98%, 99%, or 100%) identical to one or more of the following sequences:

Fc chain 1:
(SEQ ID NO: 7)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVLHEALHSHYTQKSLSLSPGK;
Fc chain 2:
(SEQ ID NO: 8)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPGK;
Conatumumab light chain:
(SEQ ID NO: 9)
EIVLTQSPGTLSLSPGERATLSCRASQGISRSYLAWYQQKPGQAPSLLI
YGASSRATGIPDRFSGSGSGTDFTLTISRLEPEDFAVYYCQQFGSSPWT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC;
Conatumumab Fab heavy chain:
(SEQ ID NO: 10)
QVQLQESGPGLVKPSQTLSLTCTVSGGSISSGDYFWSWIRQLPGKGLEW
IGHIHNSGTTYYNPSLKSRVTISVDTSKKQFSLRLSSVTAADTAVYYCA
RDRGGDYYYGMDVWGQGTTVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSC;

or combinations thereof.

In further aspects, the fusion protein is conjugated to or associated with a further moiety, such as a detectable moiety (e.g., a small molecule, fluorescent molecule, radioisotope, or magnetic particle), a pharmaceutical agent, a diagnostic agent, or combinations thereof and may comprise, for example, an antibody-drug conjugate.

In aspects wherein the further moiety is a detectable moiety, the detectable moiety may comprise a fluorescent protein, such as GFP, EGFP, Ametrine, and/or a flavin-based fluorescent protein, such as a LOV-protein, such as iLOV.

In aspects wherein the further moiety is a pharmaceutical agent, the pharmaceutical agent may comprise for example, a small molecule, peptide, lipid, carbohydrate, or toxin.

In typical aspects, the nanocage assembled from the fusion proteins described herein comprises from about 3 to about 100 nanocage monomers, none, some, or all of which may be provided as bipartite nanocage monomer subunits, such as from about 3, 4, 5, 6, 7, 8, 9, 10, 12, 14, 16, 18, 20, 22, 24, 26, 28, 30, 32, 34, 36, 38, 40, 42, 44, 46, 48, 50, 52, 55, 56, 58, 60, 62, 64, 66, 68, 70, 72, 74, 76, 78, 80, 82, 84, 86, 88, 90, 92, 94, 96, or 98 to about 4, 5, 6, 7, 8, 9, 10, 12, 14, 16, 18, 20, 22, 24, 26, 28, 30, 32, 34, 36, 38, 40, 42, 44, 46, 48, 50, 52, 55, 56, 58, 60, 62, 64, 66, 68, 70, 72, 74, 76, 78, 80, 82, 84, 86, 88, 90, 92, 94, 96, 98, or 100 nanocage monomers, such as 24, 32, or 60 monomers. The nanocage monomer or subunit thereof may be any known nanocage monomer, natural, synthetic, or partly synthetic and is, in aspects, selected from ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof. Typically, the nanocage monomer or subunit thereof is ferritin or apoferritin or a subunit thereof.

When apoferritin is chosen as the nanocage monomer and the nanocage monomer is chosen to be provided in subunits, typically the first and second nanocage monomer subunits interchangeably comprise the “N” and “C” regions of apoferritin. It will be understood that other nanocage monomers can be divided into bipartite subunits much like apoferritin as described herein so that the subunits self-assemble and are each amenable to fusion with a bioactive moiety.

Typically, the “N” region of apoferritin comprises or consists of a sequence at least 70% (such as at least 75%, 80%, 85%, 90%, 95%, 96%, 97%, 98%, 99%, or 100%) identical to:

(SEQ ID NO: 1)
MSSQIRQNYSTDVEAAVNSLVNLYLQASYTYLSLGFYFDRDDVALEGVS
HFFRELAEEKREGYERLLKMQNQRGGRALFQDIKKPAEDEW.

Typically, the “C” region of apoferritin comprises or consists of a sequence at least 70% (such as at least 75%, 80%, 85%, 90%, 95%, 96%, 97%, 98%, 99%, or 100%) identical to:

(SEQ ID NO: 2)
GKTPDAMKAAMALEKKLNQALLDLHALGSARTDPHLCDFLETHFLDEEV
KLIKKMGDHLTNLHRLGGPEAGLGEYLFERLTLRHD
or
(SEQ ID NO: 3)
GKTPDAMKAAMALEKKLNQALLDLHALGSARTDPHLCDFLETHFLDEEV
KLIKKMGDHLTNLHRLGGPEAGLGEYLFERLTLKHD.

In aspects, the fusion protein described herein further comprises a linker between the nanocage monomer or subunit thereof and the DR4 and/or DR5 antigen-binding moiety, much like the linker described above. Again, the linker may be flexible or rigid, but is typically flexible to allow the bioactive moiety to retain activity and to allow the nanocage monomers or subunits thereof to retain self-assembly properties. The linker is generally long enough to impart some flexibility to the fusion protein, although it will be understood that linker length will vary depending upon the nanocage monomer or subunits thereof and DR4 and/or DR5 sequences and the three-dimensional conformation of the fusion protein. Thus, the linker is typically from about 1 to about 30 amino acid residues, such as from about 1, 2, 3, 4, 5, 6, 7, 8, 9, 10, 11, 12, 13, 14, 15, 16, 17, 18, 19, 20, 21, 22, 23, 24, 25, 26, 27, 28, or 29 to about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues, such as from about 8 to about 16 amino acid residues, such as 8, 10, or 12 amino acid residues.

The linker may be of any amino acid sequence and, in one typical example, the linker comprises a series of G and S amino acids, such as a series of GS repeats, GGS repeats, GGGS repeats, and/or GGGGS repeats. Typically, the linker comprises a GGGGS and/or GGGS repeat and, more typically, the linker comprises at least about 2, 3, 4, 5, 6, 7, 8, 9, 10, 11, 12, 13, 14, 15, 16, 17, 18, 19, or 20 GGGS and/or GGGGS repeats, such as about 5 GGGS repeats and/or about 14 GGGGS repeats. In specific aspects, the linker comprises or consists of a sequence at least 70% (such as at least 75%, 80%, 85%, 90%, 95%, 96%, 97%, 98%, 99%, or 100%) identical to:

(SEQ ID NO: 4)
GGGGSGGGGSGGGGSGGGGSGGGGSGG.

Similarly, the fusion protein may further comprising a C-terminal linker for improving one or more attributes of the fusion protein. In aspects, the C-terminal linker, like the linkers described above, typically comprises a series of G and S amino acids, such as a series of GS repeats, GGS repeats, GGGS repeats, and/or GGGGS repeats. Typically, the linker comprises a GGS repeat and, more typically, the linker comprises about 2, 3, 4, 5, 6, 7, 8, 9, 10, 11, 12, 13, 14, 15, 16, 17, 18, 19, or 20 GGS repeats, such as about 8 GGS repeats. In specific aspects, the C-terminal linker comprises or consists of a sequence at least 70% (such as at least 75%, 80%, 85%, 90%, 95%, 96%, 97%, 98%, 99%, or 100%) identical to:

(SEQ ID NO: 5)
GGSGGSGGSGGSGGGSGGSGGSGGSG.

Also described herein are pairs of the fusion proteins described above, wherein each pair self-assembles to form a nanocage monomer, wherein the first and second nanocage monomer subunits are fused to different DR4 and/or DR5 antigen-binding moieties and/or other bioactive moieties as described herein. This provides multivalency and/or multispecificity to a single nanocage monomer assembled from the pair of subunits.

### Nanocages

Also disclosed herein are nanocages comprising at least one fusion protein as disclosed herein, wherein the nanocage self-assembles from at least one fusion protein and additional fusion protein(s) and/or nanocage monomer(s) or subunits thereof, such as ferritin chain(s) (e.g., human ferritin light chains).

Also described herein are nanocages comprising at least one fusion protein described herein and at least one nanocage monomer or subunit thereof that self-assembles with the fusion protein to form a nanocage. Further, pairs of the fusion proteins are described herein, wherein the pair self-assembles to form a nanocage monomer and wherein the first and second nanocage monomer subunits are fused to different bioactive moieties.

It will be understood that the nanocages may self-assemble from multiple identical fusion proteins, from multiple different fusion proteins (and therefore be multivalent and/or multispecific), from a combination of fusion proteins and wild-type proteins, and any combination thereof. For example, the nanocages may be decorated internally and/or externally with at least one of the fusion proteins described herein in combination with at least one anti-DR4 and/or DR5 antibody. In some aspects, at least one DR4 and/or DR5 antigen-binding moiety and at least one Fc fragment decorate the exterior surface of the nanocage. In some aspects, at least two DR4 and/or DR5 antigen-binding moieties and at least two Fc fragments decorate the exterior surface of the nanocage.

In typical aspects, from about 20% to about 80% of the nanocage monomers or subunits thereof comprise the fusion protein described herein. In view of the modular solution described herein, the nanocages could in theory comprise up to twice as many bioactive moieties as there are monomers in the nanocage, as each nanocage monomer may be divided into two subunits, each of which can independently bind to a different bioactive moiety. It will be understood that this modularity can be harnessed to achieve any desired ratio of bioactive moieties as described herein in specific example to a 4:2:1:1 ratio of four different bioactive moieties.

In some examples, the nanocages described herein may comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 or more identical or substantially identical or functionally equivalent copies of a DR4 and/or DR5 antigen-binding moiety. In additional or alternative examples, the nanocages described herein may comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 or more identical or substantially identical or functionally equivalent copies of a bioactive moiety, such as an Fc fragment. In additional or alternative examples, the nanocages described herein may comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 different DR4 and/or DR5 antigen-binding moieties and/or other bioactive moieties. In this way, the nanocages can be multivalent and/or multispecific and the extent of this can be controlled with relative ease with the systems described herein. In some embodiments, the nanocages are both multivalent and multispecific.

In some aspects, the nanocages described herein may further comprise at least one whole nanocage monomer, optionally fused to a bioactive moiety that may be the same or different from the bioactive moiety described herein as being linked to a nanocage monomer subunit.

In some aspects, the nanocages described herein comprise a first and second fusion protein each comprising a different DR4 and/or DR5 antigen-binding moiety fused to a nanocage monomer or subunit thereof, and optionally a third fusion protein comprising a bioactive moiety, such as an Fc fragment, fused to a nanocage monomer or subunit thereof.

In some embodiments, the first, second, and third fusion proteins each comprise a DR4 and/or DR5 antigen-binding moiety or a bioactive moiety, or portions thereof, fused to N- or C-half ferritin, wherein at least one of the first, second, and third fusion proteins is fused to N-half ferritin and at least one of the first, second, and third fusion proteins is fused to C-half ferritin.

In some embodiments, the first and second fusion proteins each comprise DR4 and/or DR5 antigen-binding moieties fused to full apoferritin. Similarly, in some embodiments, the third protein comprises the bioactive moiety fused to full apoferritin. It will be understood that combinations of full nanocage monomers and subunits of nanocage monomers are contemplated for use in the modular nanocages described herein.

While the proteins can comprise any numbers or ratios of fusion proteins, in some embodiments, the nanocage described herein comprises the following three proteins, optionally in a 4:1:1 ratio:
a. a DR4 and/or DR5 antigen-binding moiety, such as a Fab, such as a single chain Fab (scFab) fragment, of Conatumumab, Tigatuzumab, Drozitumab, Lexatumumab, CM005G08, CM059H03, CM084A02, T1014A04, T1014G03, T1014A02, T1014A12, T1014B01, T1014BII, T1014F08, T1014G04, T1015A02, T1015A07, T1006F07, 42/43, 44/45 or 46/47 fused to a first full length human ferritin light chain;
b. an Fc fragment (optionally a single chain Fc (scFc) fragment) fused to a second full length human ferritin light chain;
c. a third human ferritin light chain

In some embodiments, the nanocage described herein comprises the following three proteins, optionally in a 2:1:1 ratio:
a. a first DR4 and/or DR5 antigen-binding moiety, such as an Fab fragment, of Conatumumab, Tigatuzumab, Drozitumab, Lexatumumab, CM005G08, CM059H03, CM084A02, T1014A04, T1014G03, T1014A02, T1014A12, T1014B01, T1014BII, T1014F08, T1014G04, T1015A02, T1015A07, T1006F07, 42/43, 44/45 or 46/47 fused to a first full length human ferritin light chain;
b. an Fc fragment (optionally a single chain Fc (scFc) fragment) fused to the C-half of human ferritin light chain.
c. a second DR4 and/or DR5 antigen-binding moiety, such as a Fab, such as a single chain Fab (scFab) fragment, of Conatumumab, Tigatuzumab, Drozitumab, Lexatumumab, CM005G08, CM059H03, CM084A02, T1014A04, T1014G03, T1014A02, T1014A12, T1014B01, T1014BII, T1014F08, T1014G04, T1015A02, T1015A07, T1006F07, 42/43, 44/45 or 46/47 fused the N-half of human ferritin light chain. In some embodiments, the second DR4 and/or DR5 antigen-binding moiety is the same as the first DR4 and/or DR5 moiety. In some embodiments, the second DR4 and/or DR5 antigen-binding moiety is different from the first DR4 and/or DR5 moiety.

In aspects, the nanocage described herein comprises or consists of sequences at least 70% (such as at least 75%, 80%, 85%, 90%, 95%, 96%, 97%, 98%, 99%, or 100%) identical to one or more of the following sequences, wherein the ferritin subunit is in bold and linkers are underlined. While in each of these cases the full ferritin monomer is shown (MSSQIRQNYSTDVEAAVNSLVNLYLQASYTYLSLGFYFDRDDVALEGVSHFFRELAEEKRE GYERLLKMQNQRGGRALFQDIKKPAEDEWGKTPDAMKAAMALEKKLNQALLDLHALGSAR TDPHLCDFLETHFLDEEVKLIKKMGDHLTNLHRLGGPEAGLGEYLFERLTLRHD; SEQ ID NO: 11), it will be understood that N- or C-ferritin or another monomer or part thereof could be used in its place (MSSQIRQNYSTDVEAAVNSLVNLYLQASYTYLSLGFYFDRDDVALEGVSHFFRELAEEKRE GYERLLKMQNQRGGRALFQDIKKPAEDEW (SEQ ID NO: 1) in the case of N-ferritin and GKTPDAMKAAMALEKKLNQALLDLHALGSARTDPHLCDFLETHFLDEEVKLIKKMGDHLTNL HRLGGPEAGLGEYLFERLTLRHD (SEQ ID NO: 2) in the case of C-ferritin).

Conatumumab-hFerr:
(SEQ ID NO: 12)
EIVLTQSPGTLSLSPGERATLSCRASQGISRSYLAWYQQKPGQAPSLLIYGASSRAT
GIPDRFSGSGSGTDFTLTISRLEPEDFAVYYCQQFGSSPWTFGQGTKVEIKRTVAAPSVFIF
PPSDEQLKSGTASVVCLLNNFYPREAKVQWKVDNALQSGNSQESVTEQDSKDSTYSLSST
LTLSKADYEKHKVYACEVTHQGLSSPVTKSFNRGECGGGGSGGGGSGGGGSGGGGSGG
GGSGGGGSGGGGSGGGGSGGGGSGGGGSGGGGSGGGGSGGGGGGGGSQVQLQE
SGPGLVKPSQTLSLTCTVSGGSISSGDYFWSWIRQLPGKGLEWIGHIHNSGTTYYNPSLKS
RVTISVDTSKKQFSLRLSSVTAADTAVYYCARDRGGDYYYGMDVWGQGTTVTVSSASTKG
PSVFPLAPSSKSTSGGTAALGCLVKDYFPEPVTVSWNSGALTSGVHTFPAVLQSSGLYSLS
SWVTVPSSSLGTQTYICNVNHKPSNTKVDKKVEPKSCDGGGGSGGGGGGGGSGGGGS
GGGGSGGMSSQIRQNYSTDVEAAVNSLVNLYLQASYTYLSLGFYFDRDDVALEGVSHFF
RELAEEKREGYERLLKMQNQRGGRALFQDIKKPAEDEWGKTPDAMKAAMALEKKLNQA
LLDLHALGSARTDPHLCDFLETHFLDEEVKLIKKMGDHLTNLHRLGGPEAGLGEYLFERL
TLRHD;
or
(SEQ ID NO: 13)
EIVLTQSPGTLSLSPGERATLSCRASQGISRSYLAWYQQKPGQAPSLLIYGASSRAT
GIPDRFSGSGSGTDFTLTISRLEPEDFAVYYCQQFGSSPWTFGQGTKVEIKRTVAAPSVFIF
PPSDEQLKSGTASVVCLLNNFYPREAKVQWKVDNALQSGNSQESVTEQDSKDSTYSLSST
LTLSKADYEKHKVYACEVTHQGLSSPVTKSFNRGECGGGGSGGGGSGGGGGGGGSGG
GGSGGGGSGGGGGGGGSGGGGSGGGGSGGGGSGGGGSGGGGSGGGGSQVQLQE
SGPGLVKPSQTLSLTCTVSGGSISSGDYFWSWIRQLPGKGLEWIGHIHNSGTTYYNPSLKS
RVTISVDTSKKQFSLRLSSVTAADTAVYYCARDRGGDYYYGMDVWGQGTTVTVSSASTKG
PSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDGGGGSGGGGSGGGGSGGGGS
GGGGSGGSSQIRQNYSTDVEAAVNSLVNLYLQASYTYLSLGFYFDRDDVALEGVSHFFRE
LAEEKREGYERLLKMQNQRGGRALFQDIKKPAEDEWGKTPDAMKAAMALEKKLNQALL
DLHALGSARTDPHLCDFLETHFLDEEVKLIKKMGDHLTNLHRLGGPEAGLGEYLFERLTL
RHD;
Tigatuzumab-hFerr
(SEQ ID NO: 14)
DIQMTQSPSSLSASVGDRVTITCKASQDVGTAVAWYQQKPGKAPKLLIYWASTRHT
GVPSRFSGSGSGTDFTLTISSLQPEDFATYYCQQYSSYRTFGQGTKVEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGECGGGGSGGGGSGGGGSGGGGSGG
GGSGGGGSGGGGSGGGGSGGGGSGGGGSGGGGSGGGGSGGGGSGGGGSEVQLVES
GGGLVQPGGSLRLSCAASGFTFSSYVMSWVRQAPGKGLEWVATISSGGSYTYYPDSVKG
RFTISRDNAKNTLYLQMNSLRAEDTAVYYCARRGDSMITTDYWGQGTLVTVSSASTKGPSV
FPLAPSSKSTSGGTAALGCLVKDYFPEPVTVSWNSGALTSGVHTFPAVLQSSGLYSLSSVV
TVPSSSLGTQTYICNVNHKPSNTKVDKRVEPKSCGGGGSGGGGSGGGGSGGGGSGGGG
SGGMSSQIRQNYSTDVEAAVNSLVNLYLQASYTYLSLGFYFDRDDVALEGVSHFFRELAE
EKREGYERLLKMQNQRGGRALFQDIKKPAEDEWGKTPDAMKAAMALEKKLNQALLDLH
ALGSARTDPHLCDFLETHFLDEEVKLIKKMGDHLTNLHRLGGPEAGLGEYLFERLTLRHD
or
(SEQ ID NO: 15)
DIQMTQSPSSLSASVGDRVTITCKASQDVGTAVAWYQQKPGKAPKLLIYWASTRHT
GVPSRFSGSGSGTDFTLTISSLQPEDFATYYCQQYSSYRTFGQGTKVEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGECGGGGGGGGSGGGGSGGGGSGG
GGSGGGGSGGGGSGGGGSGGGGSGGGGSGGGGSGGGGSGGGGSGGGGSEVQLVES
GGGLVQPGGSLRLSCAASGFTFSSYVMSWVRQAPGKGLEWVATISSGGSYTYYPDSVKG
RFTISRDNAKNTLYLQMNSLRAEDTAVYYCARRGDSMITTDYWGQGTLVTVSSASTKGPSV
FPLAPSSKSTSGGTAALGCLVKDYFPEPVTVSWNSGALTSGVHTFPAVLQSSGLYSLSSVV
TVPSSSLGTQTYICNVNHKPSNTKVDKRVEPKSCGGGGSGGGGSGGGGSGGGGSGGGG
SGGSSQIRQNYSTDVEAAVNSLVNLYLQASYTYLSLGFYFDRDDVALEGVSHFFRELAEE
KREGYERLLKMQNQRGGRALFQDIKKPAEDEWGKTPDAMKAAMALEKKLNQALLDLHA
LGSARTDPHLCDFLETHFLDEEVKLIKKMGDHLTNLHRLGGPEAGLGEYLFERLTLRHD;
Lexatumumab-hFerr
(SEQ ID NO: 16)
LEELTQDPAVSVALGQTVRITCQGDSLRSYYASWYQQKPGQAPVLVIYGKNNRPS
GIPDRFSGSSSGNTASLTITGAQAEDEADYYCNSRDSSGNHVVFGGGTKLTVLGQPKAAPS
VTLFPPSSEELQANKATLVCLISDFYPGAVTVAWKADSSPVKAGVETTTPSKQSNNKYAAS
SYLSLTPEQWKSHRSYSCQVTHEGSTVEKTVAPTECGGGGSGGGGSGGGGGGGGSG
GGGSGGGGSGGGGSGGGGSGGGGSGGGGSGGGGGGGGSGGGGSGGGGSEVQLV
QSGGGVERPGGSLRLSCAASGFTFDDYGMSWVRQAPGKGLEWVSGINWNGGSTGYADS
VKGRVTISRDNAKNSLYLQMNSLRAEDTAVYYCAKILGAGRGWYFDLWGKGTTVTVSSAS
TKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDGGGGSGGGGSGGGGSGGG
GSGGGGSGGMSSQIRQNYSTDVEAAVNSLVNLYLQASYTYLSLGFYFDRDDVALEGVSH
FFRELAEEKREGYERLLKMQNQRGGRALFQDIKKPAEDEWGKTPDAMKAAMALEKKLN
QALLDLHALGSARTDPHLCDFLETHFLDEEVKLIKKMGDHLTNLHRLGGPEAGLGEYLFE
RLTLRHD;
or
(SEQ ID NO: 17)
LEELTQDPAVSVALGQTVRITCQGDSLRSYYASWYQQKPGQAPVLVIYGKNNRPS
GIPDRFSGSSSGNTASLTITGAQAEDEADYYCNSRDSSGNHVVFGGGTKLTVLGQPKAAPS
VTLFPPSSEELQANKATLVCLISDFYPGAVTVAWKADSSPVKAGVETTTPSKQSNNKYAAS
SYLSLTPEQWKSHRSYSCQVTHEGSTVEKTVAPTECGGGGSGGGGSGGGGSGGGGSG
GGGSGGGGSGGGGSGGGGSGGGGSGGGGSGGGGSGGGGSGGGGSGGGGSEVQLV
QSGGGVERPGGSLRLSCAASGFTFDDYGMSWVRQAPGKGLEWVSGINWNGGSTGYADS
VKGRVTISRDNAKNSLYLQMNSLRAEDTAVYYCAKILGAGRGWYFDLWGKGTTVTVSSAS
TKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDGGGGSGGGGSGGGGSGGG
GSGGGGSGGSSQIRQNYSTDVEAAVNSLVNLYLQASYTYLSLGFYFDRDDVALEGVSHF
FRELAEEKREGYERLLKMQNQRGGRALFQDIKKPAEDEWGKTPDAMKAAMALEKKLNQ
ALLDLHALGSARTDPHLCDFLETHFLDEEVKLIKKMGDHLTNLHRLGGPEAGLGEYLFER
LTLRHD;
Drozitumab-hFerr
(SEQ ID NO: 18)
SELTQDPAVSVALGQTVRITCSGDSLRSYYASWYQQKPGQAPVLVIYGANNRPSGI
PDRFSGSSSGNTASLTITGAQAEDEADYYCNSADSSGNHVVFGGGTKLTVLGQPKAAPSV
TLFPPSSEELQANKATLVCLISDFYPGAVTVAWKADSSPVKAGVETTTPSKQSNNKYAASS
YLSLTPEQWKSHKSYSCQVTHEGSTVEKTVAPTECGGGGSGGGGSGGGGSGGGGSGG
GGSGGGGSGGGGSGGGGSGGGGSGGGGGGGGSGGGGSGGGGSGGGGSEVQLVQS
GGGVERPGGSLRLSCAASGFTFDDYAMSWVRQAPGKGLEWVSGINWQGGSTGYADSVK
GRVTISRDNAKNSLYLQMNSLRAEDTAVYYCAKILGAGRGWYFDYWGKGTTVTVSSASTK
GPSVFPLAPSSKSTSGGTAALGCLVKDYFPEPVTVSWNSGALTSGVHTFPAVLQSSGLYSL
SSWVTVPSSSLGTQTYICNVNHKPSNTKVDKKVEPKSCGGGGSGGGGSGGGGSGGGGS
GGGGSGGMSSQIRQNYSTDVEAAVNSLVNLYLQASYTYLSLGFYFDRDDVALEGVSHFF
RELAEEKREGYERLLKMQNQRGGRALFQDIKKPAEDEWGKTPDAMKAAMALEKKLNQA
LLDLHALGSARTDPHLCDFLETHFLDEEVKLIKKMGDHLTNLHRLGGPEAGLGEYLFERL
TLRHD;
or
(SEQ ID NO: 19)
SELTQDPAVSVALGQTVRITCSGDSLRSYYASWYQQKPGQAPVLVIYGANNRPSGI
PDRFSGSSSGNTASLTITGAQAEDEADYYCNSADSSGNHVVFGGGTKLTVLGQPKAAPSV
TLFPPSSEELQANKATLVCLISDFYPGAVTVAWKADSSPVKAGVETTTPSKQSNNKYAASS
YLSLTPEQWKSHKSYSCQVTHEGSTVEKTVAPTECGGGGSGGGGSGGGGSGGGGSGG
GGSGGGGSGGGGSGGGGSGGGGGGGGSGGGGSGGGGSGGGGGGGGSEVQLVQS
GGGVERPGGSLRLSCAASGFTFDDYAMSWVRQAPGKGLEWVSGINWQGGSTGYADSVK
GRVTISRDNAKNSLYLQMNSLRAEDTAVYYCAKILGAGRGWYFDYWGKGTTVTVSSASTK
GPSVFPLAPSSKSTSGGTAALGCLVKDYFPEPVTVSWNSGALTSGVHTFPAVLQSSGLYSL
SSWVTVPSSSLGTQTYICNVNHKPSNTKVDKKVEPKSCGGGGSGGGGSGGGGSGGGGS
GGGGSGGSSQIRQNYSTDVEAAVNSLVNLYLQASYTYLSLGFYFDRDDVALEGVSHFFRE
LAEEKREGYERLLKMQNQRGGRALFQDIKKPAEDEWGKTPDAMKAAMALEKKLNQALL
DLHALGSARTDPHLCDFLETHFLDEEVKLIKKMGDHLTNLHRLGGPEAGLGEYLFERLTL
RHD;
CM005G08-hFerr
(SEQ ID NO: 20)
ELTQDPAVSVALGQTVRITCQGDSLRSYYASWYQQKPGQAPVLVIYGKNNRPSGIP
DRFSGSSSGNTASLTITGAQAEDEADYYCNSRDSSGNHVVFGGGTKLTVLGQPKAAPSVT
LFPPSSEELQANKATLVCLISDFYPGAVTVAWKADSSPVKAGVETTTPSKQSNNKYAASSY
LSLTPEQWKSHRSYSCQVTHEGSTVEKTVAPTECGGGGSGGGGSGGGGSGGGGSGGG
GSGGGGSGGGGSGGGGSGGGGSGGGGSGGGGSGGGGSGGGGSGGGGSEVQLVQSG
GGVERPGGSLRLSCAASGFTFDDYGMSWVRQAPGKGLEWVSGINWNGGSTGYADSVKG
RVTISRDNAKNSLYLQMNSLRAEDTAVYYCAKILGAGRGWYFDLWGKGTTVTVSSASTKG
PSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDGGGGSGGGGSGGGGSGGGGS
GGGGSGGMSSQIRQNYSTDVEAAVNSLVNLYLQASYTYLSLGFYFDRDDVALEGVSHFF
RELAEEKREGYERLLKMQNQRGGRALFQDIKKPAEDEWGKTPDAMKAAMALEKKLNQA
LLDLHALGSARTDPHLCDFLETHFLDEEVKLIKKMGDHLTNLHRLGGPEAGLGEYLFERL
TLRHD;
or
(SEQ ID NO: 21)
ELTQDPAVSVALGQTVRITCQGDSLRSYYASWYQQKPGQAPVLVIYGKNNRPSGIP
DRFSGSSSGNTASLTITGAQAEDEADYYCNSRDSSGNHVVFGGGTKLTVLGQPKAAPSVT
LFPPSSEELQANKATLVCLISDFYPGAVTVAWKADSSPVKAGVETTTPSKQSNNKYAASSY
LSLTPEQWKSHRSYSCQVTHEGSTVEKTVAPTECGGGGSGGGGSGGGGSGGGGSGGG
GSGGGGSGGGGGGGGSGGGGSGGGGSGGGGSGGGGSGGGGSGGGGSEVQLVQSG
GGVERPGGSLRLSCAASGFTFDDYGMSWVRQAPGKGLEWVSGINWNGGSTGYADSVKG
RVTISRDNAKNSLYLQMNSLRAEDTAVYYCAKILGAGRGWYFDLWGKGTTVTVSSASTKG
PSVFPLAPSSKSTSGGTAALGCLVKDYFPEPVTVSWNSGALTSGVHTFPAVLQSSGLYSLS
SWVTVPSSSLGTQTYICNVNHKPSNTKVDKKVEPKSCDGGGGSGGGGSGGGGGGGGS
GGGGSGGSSQIRQNYSTDVEAAVNSLVNLYLQASYTYLSLGFYFDRDDVALEGVSHFFRE
LAEEKREGYERLLKMQNQRGGRALFQDIKKPAEDEWGKTPDAMKAAMALEKKLNQALL
DLHALGSARTDPHLCDFLETHFLDEEVKLIKKMGDHLTNLHRLGGPEAGLGEYLFERLTL
RHD;
CM059H03-hFerr
(SEQ ID NO: 22)
ALETTLTQSPGTLSLSPGERATLSCRASQSISSSNLAWYQQKPGRAPRLLIYGASSR
AIGIPDRFSGSGSGTDFTLTISRLEAEDFAVYYCQQYGSSPITFGQGTRLEIKRTVAAPSVFIF
PPSDEQLKSGTASVVCLLNNFYPREAKVQWKVDNALQSGNSQESVTEQDSKDSTYSLSST
LTLSKADYEKHKVYACEVTHQGLSSPVTKSFNRGECGGGGSGGGGSGGGGSGGGGSGG
GGSGGGGSGGGGGGGGSGGGGSGGGGSGGGGSGGGGSGGGGSGGGGSEVQLVQS
GAEVKKPGASVKVSCRASGYTFTSYGITWVRQAPGQGLEWMGWISAYNGKTNYVQELQG
RVTMTTDTSTSTVYMELTSLRSDDTAVYYCARRGNNYRFGYFDFWGQGTLVTVSSASTKG
PSVFPLAPSSKSTSGGTAALGCLVKDYFPEPVTVSWNSGALTSGVHTFPAVLQSSGLYSLS
SWVTVPSSSLGTQTYICNVNHKPSNTKVDKKVEPKSCDGGGGGGGGSGGGGSGGGGS
GGGGSGGMSSQIRQNYSTDVEAAVNSLVNLYLQASYTYLSLGFYFDRDDVALEGVSHFF
RELAEEKREGYERLLKMQNQRGGRALFQDIKKPAEDEWGKTPDAMKAAMALEKKLNQA
LLDLHALGSARTDPHLCDFLETHFLDEEVKLIKKMGDHLTNLHRLGGPEAGLGEYLFERL
TLRHD;
or
(SEQ ID NO: 23)
ALETTLTQSPGTLSLSPGERATLSCRASQSISSSNLAWYQQKPGRAPRLLIYGASSR
AIGIPDRFSGSGSGTDFTLTISRLEAEDFAVYYCQQYGSSPITFGQGTRLEIKRTVAAPSVFIF
PPSDEQLKSGTASVVCLLNNFYPREAKVQWKVDNALQSGNSQESVTEQDSKDSTYSLSST
LTLSKADYEKHKVYACEVTHQGLSSPVTKSFNRGECGGGGSGGGGSGGGGSGGGGSGG
GGSGGGGSGGGGSGGGGSGGGGSGGGGSGGGGSGGGGSGGGGSGGGGSEVQLVQS
GAEVKKPGASVKVSCRASGYTFTSYGITWVRQAPGQGLEWMGWISAYNGKTNYVQELQG
RVTMTTDTSTSTVYMELTSLRSDDTAVYYCARRGNNYRFGYFDFWGQGTLVTVSSASTKG
PSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDGGGGSGGGGSGGGGSGGGGS
GGGGSGGSSQIRQNYSTDVEAAVNSLVNLYLQASYTYLSLGFYFDRDDVALEGVSHFFRE
LAEEKREGYERLLKMQNQRGGRALFQDIKKPAEDEWGKTPDAMKAAMALEKKLNQALL
DLHALGSARTDPHLCDFLETHFLDEEVKLIKKMGDHLTNLHRLGGPEAGLGEYLFERLTL
RHD;
CM084A02-hFerr
(SEQ ID NO: 24)
AQSVLTQPPSASGTPGQRVSISCSGSSSNIGSNTVIWYQQLPGTAPKLLMYSNDRR
PSGVPDRFSGSKSGTSASLAISGLQSEDEADYYCATWDDSLNGHYVFGTGTKLTVLGQPK
AAPSVTLFPPSSEELQANKATLVCLISDFYPGAVTVAWKADSSPVKAGVETTTPSKQSNNK
YAASSYLSLTPEQWKSHRSYSCQVTHEGSTVEKTVAPTECGGGGGGGGSGGGGSGGG
GSGGGGSGGGGSGGGGSGGGGSGGGGSGGGGSGGGGSGGGGSGGGGSGGGGSEV
QLVQSGAEVKKPGASVKLSCKASGYTLVNYFMHWVRQAPGQGPEWMGMINPSGGTTKN
RQKFQDRVTMTRDTSTRTVYMELSGLTSEDTAVYYCATDFKGTDILFRDWGRGTLVTVSS
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDGGGGSGGGGSGGGGSG
GGGSGGGGSGGMSSQIRQNYSTDVEAAVNSLVNLYLQASYTYLSLGFYFDRDDVALEGV
SHFFRELAEEKREGYERLLKMQNQRGGRALFQDIKKPAEDEWGKTPDAMKAAMALEKK
LNQALLDLHALGSARTDPHLCDFLETHFLDEEVKLIKKMGDHLTNLHRLGGPEAGLGEYL
FERLTLRHD;
or
(SEQ ID NO: 25)
AQSVLTQPPSASGTPGQRVSISCSGSSSNIGSNTVIWYQQLPGTAPKLLMYSNDRR
PSGVPDRFSGSKSGTSASLAISGLQSEDEADYYCATWDDSLNGHYVFGTGTKLTVLGQPK
AAPSVTLFPPSSEELQANKATLVCLISDFYPGAVTVAWKADSSPVKAGVETTTPSKQSNNK
YAASSYLSLTPEQWKSHRSYSCQVTHEGSTVEKTVAPTECGGGGSGGGGSGGGGSGGG
GSGGGGSGGGGSGGGGGGGGSGGGGSGGGGSGGGGSGGGGSGGGGSGGGGSEV
QLVQSGAEVKKPGASVKLSCKASGYTLVNYFMHWVRQAPGQGPEWMGMINPSGGTTKN
RQKFQDRVTMTRDTSTRTVYMELSGLTSEDTAVYYCATDFKGTDILFRDWGRGTLVTVSS
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDGGGGSGGGGSGGGGSG
GGGSGGGGSGGSSQIRQNYSTDVEAAVNSLVNLYLQASYTYLSLGFYFDRDDVALEGVS
HFFRELAEEKREGYERLLKMQNQRGGRALFQDIKKPAEDEWGKTPDAMKAAMALEKKL
NQALLDLHALGSARTDPHLCDFLETHFLDEEVKLIKKMGDHLTNLHRLGGPEAGLGEYLF
ERLTLRHD;
T1014A04-hFerr
(SEQ ID NO: 26)
AQSVLTQPPSASGSPGQSVTISCTGTTSDVGGYNYVSWYQQHPGKAPKLMIYGVN
QRPSGVPDRFSGSKSGNTASLTVSGLQAEDEADYYCSSYAGSNNWVFGGGTKVTVLGQP
KAAPSVTLFPPSSEELQANKATLVCLISDFYPGAVTVAWKADSSPVKAGVETTTPSKQSNN
KYAASSYLSLTPEQWKSHRSYSCQVTHEGSTVEKTVAPTECGGGGSGGGGSGGGGSGG
GGSGGGGSGGGGSGGGGSGGGGSGGGGSGGGGSGGGGSGGGGSGGGGGGGGSE
VQLVQSGADVKRPGASVKVSCKISGDSFNAYFIHWVRQAPGQGLEWMGWFNPDSGTADS
AQKFHGRVTMTRDTSSSTAFLELSRLRSDDTAVYYCVRQHRGNTFAPWGRGTMVTVSSA
STKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDGGGGGGGGSGGGGSGG
GGSGGGGSGGMSSQIRQNYSTDVEAAVNSLVNLYLQASYTYLSLGFYFDRDDVALEGVS
HFFRELAEEKREGYERLLKMQNQRGGRALFQDIKKPAEDEWGKTPDAMKAAMALEKKL
NQALLDLHALGSARTDPHLCDFLETHFLDEEVKLIKKMGDHLTNLHRLGGPEAGLGEYLF
ERLTLRHD;
or
(SEQ ID NO: 27)
AQSVLTQPPSASGSPGQSVTISCTGTTSDVGGYNYVSWYQQHPGKAPKLMIYGVN
QRPSGVPDRFSGSKSGNTASLTVSGLQAEDEADYYCSSYAGSNNWVFGGGTKVTVLGQP
KAAPSVTLFPPSSEELQANKATLVCLISDFYPGAVTVAWKADSSPVKAGVETTTPSKQSNN
KYAASSYLSLTPEQWKSHRSYSCQVTHEGSTVEKTVAPTECGGGGSGGGGSGGGGSGG
GGSGGGGGGGGSGGGGSGGGGSGGGGSGGGGSGGGGSGGGGSGGGGSGGGGSE
VQLVQSGADVKRPGASVKVSCKISGDSFNAYFIHWVRQAPGQGLEWMGWFNPDSGTADS
AQKFHGRVTMTRDTSSSTAFLELSRLRSDDTAVYYCVRQHRGNTFAPWGRGTMVTVSSA
STKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDGGGGSGGGGSGGGGSGG
GGSGGGGSGGSSQIRQNYSTDVEAAVNSLVNLYLQASYTYLSLGFYFDRDDVALEGVSH
FFRELAEEKREGYERLLKMQNQRGGRALFQDIKKPAEDEWGKTPDAMKAAMALEKKLN
QALLDLHALGSARTDPHLCDFLETHFLDEEVKLIKKMGDHLTNLHRLGGPEAGLGEYLFE
RLTLRHD;
T1014G03-hFerr
(SEQ ID NO: 28)
AQSALTQPASVSGSPGQSITISCTGTSSDIGAYKYVSWYQQHPGKAPKLVIYEVSNR
PSGVSSRFSGSKSGQTASLTISGLQADDEADYYCNSYQGYNTWVFGGGTKVTVLGQPKAA
PSVTLFPPSSEELQANKATLVCLISDFYPGAVTVAWKADSSPVKAGVETTTPSKQSNNKYA
ASSYLSLTPEQWKSHRSYSCQVTHEGSTVEKTVAPTECGGGGSGGGGSGGGGSGGGGS
GGGGSGGGGSGGGGSGGGGSGGGGSGGGGSGGGGSGGGGSGGGGSGGGGSEVQL
VQSGAEVKMPGASVKLSCRVSGDTFTAYFIHWVRQAPGQGLEWMGWFNPISGTAGSAEK
FRGRVAMTRDTSISTAYMELNRLTFDDTAVYYCARQHRGNTFDPWGQGTLVTVSSASTKG
PSVFPLAPSSKSTSGGTAALGCLVKDYFPEPVTVSWNSGALTSGVHTFPAVLQSSGLYSLS
SWVTVPSSSLGTQTYICNVNHKPSNTKVDKKVEPKSCDGGGGSGGGGSGGGGSGGGGS
GGGGSGGMSSQIRQNYSTDVEAAVNSLVNLYLQASYTYLSLGFYFDRDDVALEGVSHFF
RELAEEKREGYERLLKMQNQRGGRALFQDIKKPAEDEWGKTPDAMKAAMALEKKLNQA
LLDLHALGSARTDPHLCDFLETHFLDEEVKLIKKMGDHLTNLHRLGGPEAGLGEYLFERL
TLRHD;
or
(SEQ ID NO: 29)
AQSALTQPASVSGSPGQSITISCTGTSSDIGAYKYVSWYQQHPGKAPKLVIYEVSNR
PSGVSSRFSGSKSGQTASLTISGLQADDEADYYCNSYQGYNTWVFGGGTKVTVLGQPKAA
PSVTLFPPSSEELQANKATLVCLISDFYPGAVTVAWKADSSPVKAGVETTTPSKQSNNKYA
ASSYLSLTPEQWKSHRSYSCQVTHEGSTVEKTVAPTECGGGGSGGGGSGGGGSGGGGS
GGGGSGGGGSGGGGSGGGGSGGGGSGGGGSGGGGSGGGGSGGGGSGGGGSEVQL
VQSGAEVKMPGASVKLSCRVSGDTFTAYFIHWVRQAPGQGLEWMGWFNPISGTAGSAEK
FRGRVAMTRDTSISTAYMELNRLTFDDTAVYYCARQHRGNTFDPWGQGTLVTVSSASTKG
PSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDGGGGSGGGGSGGGGSGGGGS
GGGGSGGMSSQIRQNYSTDVEAAVNSLVNLYLQASYTYLSLGFYFDRDDVALEGVSHFF
RELAEEKREGYERLLKMQNQRGGRALFQDIKKPAEDEWGKTPDAMKAAMALEKKLNQA
LLDLHALGSARTDPHLCDFLETHFLDEEVKLIKKMGDHLTNLHRLGGPEAGLGEYLFERL
TLRHD;
T1014A02-hFerr
(SEQ ID NO: 30)
ALSYVLTQPPSASGTPGQRVTISCAGSSSNIGGNTVNWYQQLPATAPKLLIYSNNQ
RPSGVPDRFSGSKSGTSASLAISGLQSEDEADYYCATWDDSRGGWVFGGGTKLTVLGQP
KAAPSVTLFPPSSEELQANKATLVCLISDFYPGAVTVAWKADSSPVKAGVETTTPSKQSNN
KYAASSYLSLTPEQWKSHRSYSCQVTHEGSTVEKTVAPTECGGGGSGGGGSGGGGSGG
GGSGGGGSGGGGSGGGGSGGGGSGGGGSGGGGSGGGGSGGGGSGGGGSGGGGSQ
VQLQESGPGLVKPSETLSLTCTVSGGSISDYYWSWVRQSPGKGLEWIGSIDYAGSTNYNP
SLKSRVTMTIDKSKKQFPLKIDSVTAADTAMYYCARQLGRISDYWGQGTLVTVSSASTKGP
SVFPLAPSSKSTSGGTAALGCLVKDYFPEPVTVSWNSGALTSGVHTFPAVLQSSGLYSLSS
WVTVPSSSLGTQTYICNVNHKPSNTKVDKKVEPKSCDGGGGSGGGGSGGGGSGGGGSG
GGGSGGMSSQIRQNYSTDVEAAVNSLVNLYLQASYTYLSLGFYFDRDDVALEGVSHFFR
ELAEEKREGYERLLKMQNQRGGRALFQDIKKPAEDEWGKTPDAMKAAMALEKKLNQAL
LDLHALGSARTDPHLCDFLETHFLDEEVKLIKKMGDHLTNLHRLGGPEAGLGEYLFERLT
LRHD;
or
(SEQ ID NO: 31)
ALSYVLTQPPSASGTPGQRVTISCAGSSSNIGGNTVNWYQQLPATAPKLLIYSNNQ
RPSGVPDRFSGSKSGTSASLAISGLQSEDEADYYCATWDDSRGGWVFGGGTKLTVLGQP
KAAPSVTLFPPSSEELQANKATLVCLISDFYPGAVTVAWKADSSPVKAGVETTTPSKQSNN
KYAASSYLSLTPEQWKSHRSYSCQVTHEGSTVEKTVAPTECGGGGSGGGGSGGGGSGG
GGSGGGGSGGGGSGGGGSGGGGSGGGGSGGGGSGGGGSGGGGSGGGGSGGGGSQ
VQLQESGPGLVKPSETLSLTCTVSGGSISDYYWSWVRQSPGKGLEWIGSIDYAGSTNYNP
SLKSRVTMTIDKSKKQFPLKIDSVTAADTAMYYCARQLGRISDYWGQGTLVTVSSASTKGP
SVFPLAPSSKSTSGGTAALGCLVKDYFPEPVTVSWNSGALTSGVHTFPAVLQSSGLYSLSS
WTVPSSSLGTQTYICNVNHKPSNTKVDKKVEPKSCDGGGGSGGGGSGGGGSGGGGSG
GGGSGGSSQIRQNYSTDVEAAVNSLVNLYLQASYTYLSLGFYFDRDDVALEGVSHFFREL
AEEKREGYERLLKMQNQRGGRALFQDIKKPAEDEWGKTPDAMKAAMALEKKLNQALLD
LHALGSARTDPHLCDFLETHFLDEEVKLIKKMGDHLTNLHRLGGPEAGLGEYLFERLTLR
HD;
T1014A12-hFerr
(SEQ ID NO: 32)
AQSALTQPASVSGPPGQSITISCTGSSSDVGGYKYVSWYQQHPGKAPKLIIHDVSR
RPSEVSSRFSGSKSGNTASLTISGLQAEDEAEYYCSSYSSTNSWVFGGGTKVTVLGQPKA
APSVTLFPPSSEELQANKATLVCLISDFYPGAVTVAWKADSSPVKAGVETTTPSKQSNNKY
AASSYLSLTPEQWKSHRSYSCQVTHEGSTVEKTVAPTECGGGGSGGGGGGGGSGGGG
SGGGGSGGGGSGGGGSGGGGSGGGGSGGGGSGGGGSGGGGSGGGGSGGGGSEVQ
LVQSGADVKRPGASVKVSCKISGDSFTAYFIHWVRQAPGQGLEWMGWFNPDSGTADSAQ
KFHGRVTMTRDTSSSTAFLELSRLRSDDTAVYYCVRQHRGNTFAPWGRGTMVTVSSASTK
GPSVFPLAPSSKSTSGGTAALGCLVKDYFPEPVTVSWNSGALTSGVHTFPAVLQSSGLYSL
SSWTVPSSSLGTQTYICNVNHKPSNTKVDKKVEPKSCDGGGGSGGGGSGGGGSGGGG
SGGGGSGGMSSQIRQNYSTDVEAAVNSLVNLYLQASYTYLSLGFYFDRDDVALEGVSHF
FRELAEEKREGYERLLKMQNQRGGRALFQDIKKPAEDEWGKTPDAMKAAMALEKKLNQ
ALLDLHALGSARTDPHLCDFLETHFLDEEVKLIKKMGDHLTNLHRLGGPEAGLGEYLFER
LTLRHD;
or
(SEQ ID NO: 33)
AQSALTQPASVSGPPGQSITISCTGSSSDVGGYKYVSWYQQHPGKAPKLIIHDVSR
RPSEVSSRFSGSKSGNTASLTISGLQAEDEAEYYCSSYSSTNSWVFGGGTKVTVLGQPKA
APSVTLFPPSSEELQANKATLVCLISDFYPGAVTVAWKADSSPVKAGVETTTPSKQSNNKY
AASSYLSLTPEQWKSHRSYSCQVTHEGSTVEKTVAPTECGGGGSGGGGGGGGSGGGG
SGGGGSGGGGSGGGGSGGGGSGGGGSGGGGSGGGGSGGGGSGGGGSGGGGSEVQ
LVQSGADVKRPGASVKVSCKISGDSFTAYFIHWVRQAPGQGLEWMGWFNPDSGTADSAQ
KFHGRVTMTRDTSSSTAFLELSRLRSDDTAVYYCVRQHRGNTFAPWGRGTMVTVSSASTK
GPSVFPLAPSSKSTSGGTAALGCLVKDYFPEPVTVSWNSGALTSGVHTFPAVLQSSGLYSL
SSWVTVPSSSLGTQTYICNVNHKPSNTKVDKKVEPKSCDGGGGSGGGGSGGGGSGGGG
SGGGGSGGSSQIRQNYSTDVEAAVNSLVNLYLQASYTYLSLGFYFDRDDVALEGVSHFFR
ELAEEKREGYERLLKMQNQRGGRALFQDIKKPAEDEWGKTPDAMKAAMALEKKLNQAL
LDLHALGSARTDPHLCDFLETHFLDEEVKLIKKMGDHLTNLHRLGGPEAGLGEYLFERLT
LRHD;
T1014B01-hFerr
(SEQ ID NO: 34)
AQSVVTQPPSVSGSPGQSVTISCTGTSSDIGAYNYVSWFQQHPGKAPKLIISEVSKR
PSGVPDRLSGSKSGNTASLTVSGLQAEDEADYYCGSYAGSNIWVFGGGTKVTVLG
QPKAAPSVTLFPPSSEELQANKATLVCLISDFYPGAVTVAWKADSSPVKAGVETTTPSKQS
NNKYAASSYLSLTPEQWKSHRSYSCQVTHEGSTVEKTVAPTECGGGGSGGGGSGGGGS
GGGGSGGGGSGGGGSGGGGSGGGGSGGGGSGGGGGGGGSGGGGSGGGGSGGGG
SQVQLVQSGAEVKKPGASVKVSCKISGDTFAAYFIHWVRQAPGQGLEWMGWFNPNSGTA
DSSQKFHGRVTMTRDTSISTAYMELSRLRSDDTAVYYCARQHRSNTFDPWGQGTMVTVS
SASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDGGGGSGGGGSGGGGS
GGGGSGGGGSGGMSSQIRQNYSTDVEAAVNSLVNLYLQASYTYLSLGFYFDRDDVALEG
VSHFFRELAEEKREGYERLLKMQNQRGGRALFQDIKKPAEDEWGKTPDAMKAAMALEK
KLNQALLDLHALGSARTDPHLCDFLETHFLDEEVKLIKKMGDHLTNLHRLGGPEAGLGEY
LFERLTLRHD;
or
(SEQ ID NO: 35)
AQSVVTQPPSVSGSPGQSVTISCTGTSSDIGAYNYVSWFQQHPGKAPKLIISEVSKR
PSGVPDRLSGSKSGNTASLTVSGLQAEDEADYYCGSYAGSNIWVFGGGTKVTVLG
QPKAAPSVTLFPPSSEELQANKATLVCLISDFYPGAVTVAWKADSSPVKAGVETTTPSKQS
NNKYAASSYLSLTPEQWKSHRSYSCQVTHEGSTVEKTVAPTECGGGGSGGGGSGGGGS
GGGGSGGGGSGGGGSGGGGSGGGGSGGGGSGGGGSGGGGSGGGGSGGGGSGGGG
SQVQLVQSGAEVKKPGASVKVSCKISGDTFAAYFIHWVRQAPGQGLEWMGWFNPNSGTA
DSSQKFHGRVTMTRDTSISTAYMELSRLRSDDTAVYYCARQHRSNTFDPWGQGTMVTVS
SASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDGGGGSGGGGSGGGGS
GGGGSGGGGSGGSSQIRQNYSTDVEAAVNSLVNLYLQASYTYLSLGFYFDRDDVALEGV
SHFFRELAEEKREGYERLLKMQNQRGGRALFQDIKKPAEDEWGKTPDAMKAAMALEKK
LNQALLDLHALGSARTDPHLCDFLETHFLDEEVKLIKKMGDHLTNLHRLGGPEAGLGEYL
FERLTLRHD;
T1014Bll-hFerr
(SEQ ID NO: 36)
AQSALTQPASVSGSPGQSITISCTGTNSDVGGYNYVSWYQQHPGKAPKLMIYEVN
NRPSGVSNRFSGSKSGNTASLTISGLQADDEADYYCSSYTTSNTWVFGGGTKLTVLGQPK
AAPSVTLFPPSSEELQANKATLVCLISDFYPGAVTVAWKADSSPVKAGVETTTPSKQSNNK
YAASSYLSLTPEQWKSHRSYSCQVTHEGSTVEKTVAPTECGGGGSGGGGSGGGGSGGG
GSGGGGSGGGGSGGGGSGGGGSGGGGSGGGGSGGGGSGGGGSGGGGSGGGGSEV
QLVQSGAEVKKPGASVKVSCKISGDSFTAYFIHWLRQAPGEGLEWMGWFNPISGTAGSPQ
KFHGRVAMTRDTSISTAYMELTRLASDDTAIYYCARQHHSNTFDPWGQGTLVTVSSASTKG
PSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDGGGGSGGGGSGGGGSGGGGS
GGGGSGGMSSQIRQNYSTDVEAAVNSLVNLYLQASYTYLSLGFYFDRDDVALEGVSHFF
RELAEEKREGYERLLKMQNQRGGRALFQDIKKPAEDEWGKTPDAMKAAMALEKKLNQA
LLDLHALGSARTDPHLCDFLETHFLDEEVKLIKKMGDHLTNLHRLGGPEAGLGEYLFERL
TLRHD;
or
(SEQ ID NO: 37)
AQSALTQPASVSGSPGQSITISCTGTNSDVGGYNYVSWYQQHPGKAPKLMIYEVN
NRPSGVSNRFSGSKSGNTASLTISGLQADDEADYYCSSYTTSNTWVFGGGTKLTVLGQPK
AAPSVTLFPPSSEELQANKATLVCLISDFYPGAVTVAWKADSSPVKAGVETTTPSKQSNNK
YAASSYLSLTPEQWKSHRSYSCQVTHEGSTVEKTVAPTECGGGGSGGGGSGGGGSGGG
GSGGGGSGGGGSGGGGSGGGGSGGGGSGGGGSGGGGSGGGGSGGGGSGGGGSEV
QLVQSGAEVKKPGASVKVSCKISGDSFTAYFIHWLRQAPGEGLEWMGWFNPISGTAGSPQ
KFHGRVAMTRDTSISTAYMELTRLASDDTAIYYCARQHHSNTFDPWGQGTLVTVSSASTKG
PSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDGGGGSGGGGSGGGGSGGGGS
GGGGSGGSSQIRQNYSTDVEAAVNSLVNLYLQASYTYLSLGFYFDRDDVALEGVSHFFRE
LAEEKREGYERLLKMQNQRGGRALFQDIKKPAEDEWGKTPDAMKAAMALEKKLNQALL
DLHALGSARTDPHLCDFLETHFLDEEVKLIKKMGDHLTNLHRLGGPEAGLGEYLFERLTL
RHD;
T1014F08-hFerr
(SEQ ID NO: 38)
ALPVLTQPPSASGSPGQSVTISCTGTSSDVGGYKYVSWYQQHPGKAPKLMIYEVS
MRPSGVPDRFSGSKSGNTASLTVSGLQAEDEADYYCASYAGSNNWVFGGGTKLTVLGQP
KAAPSVTLFPPSSEELQANKATLVCLISDFYPGAVTVAWKADSSPVKAGVETTTPSKQSNN
KYAASSYLSLTPEQWKSHRSYSCQVTHEGSTVEKTVAPTECGGGGSGGGGSGGGGSGG
GGSGGGGSGGGGSGGGGSGGGGSGGGGSGGGGSGGGGSGGGGSGGGGSGGGGSE
VQLVQSGAEVKKPGASVKLSCRVSGDTFTAYFIHWVRQAPGQGPEWMGWFNPISGTAGS
AARFRGRVAMTRDTSISTAYMELNRLTFDDTAVYYCARQHRGNTFDPWGKGTLVTVSSAS
TKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDGGGGSGGGGSGGGGSGGG
GSGGGGSGGMSSQIRQNYSTDVEAAVNSLVNLYLQASYTYLSLGFYFDRDDVALEGVSH
FFRELAEEKREGYERLLKMQNQRGGRALFQDIKKPAEDEWGKTPDAMKAAMALEKKLN
QALLDLHALGSARTDPHLCDFLETHFLDEEVKLIKKMGDHLTNLHRLGGPEAGLGEYLFE
RLTLRHD;
or
(SEQ ID NO: 39)
ALPVLTQPPSASGSPGQSVTISCTGTSSDVGGYKYVSWYQQHPGKAPKLMIYEVS
MRPSGVPDRFSGSKSGNTASLTVSGLQAEDEADYYCASYAGSNNWVFGGGTKLTVLGQP
KAAPSVTLFPPSSEELQANKATLVCLISDFYPGAVTVAWKADSSPVKAGVETTTPSKQSNN
KYAASSYLSLTPEQWKSHRSYSCQVTHEGSTVEKTVAPTECGGGGSGGGGSGGGGSGG
GGSGGGGGGGGSGGGGSGGGGSGGGGSGGGGSGGGGSGGGGSGGGGSGGGGSE
VQLVQSGAEVKKPGASVKLSCRVSGDTFTAYFIHWVRQAPGQGPEWMGWFNPISGTAGS
AARFRGRVAMTRDTSISTAYMELNRLTFDDTAVYYCARQHRGNTFDPWGKGTLVTVSSAS
TKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDGGGGSGGGGSGGGGSGGG
GSGGGGSGGSSQIRQNYSTDVEAAVNSLVNLYLQASYTYLSLGFYFDRDDVALEGVSHF
FRELAEEKREGYERLLKMQNQRGGRALFQDIKKPAEDEWGKTPDAMKAAMALEKKLNQ
ALLDLHALGSARTDPHLCDFLETHFLDEEVKLIKKMGDHLTNLHRLGGPEAGLGEYLFER
LTLRHD;
T1014G04-hFerr
(SEQ ID NO: 40)
AQPVLTQPPSASGSPGQSVTISCTGTSSDVGSYEYVSWYQQHPGKAPRLMISEVN
KRPSGVPNRFSGSKSGNTASLTVSGLQADDEADYYCSSYAGSNNWVFGGGTKVTVLGQP
KAAPSVTLFPPSSEELQANKATLVCLISDFYPGAVTVAWKADSSPVKAGVETTTPSKQSNN
KYAASSYLSLTPEQWKSHRSYSCQVTHEGSTVEKTVAPTECGGGGSGGGGSGGGGSGG
GGSGGGGSGGGGSGGGGSGGGGSGGGGSGGGGGGGGSGGGGSGGGGSGGGGSE
VQLVQSGADVKRPGASVKVSCKISGDSFTAYFIHWVRQAPGQGLEWMGWFNPDSGTADS
AQKFHGRVTMTRDTSSSTAFLELSRLRSDDTAVYYCVRQHRGNTFAPWGRGTLVTVSSAS
TKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDGGGGSGGGGSGGGGSGGG
GSGGGGSGGMSSQIRQNYSTDVEAAVNSLVNLYLQASYTYLSLGFYFDRDDVALEGVSH
FFRELAEEKREGYERLLKMQNQRGGRALFQDIKKPAEDEWGKTPDAMKAAMALEKKLN
QALLDLHALGSARTDPHLCDFLETHFLDEEVKLIKKMGDHLTNLHRLGGPEAGLGEYLFE
RLTLRHD;
or
(SEQ ID NO: 41)
AQPVLTQPPSASGSPGQSVTISCTGTSSDVGSYEYVSWYQQHPGKAPRLMISEVN
KRPSGVPNRFSGSKSGNTASLTVSGLQADDEADYYCSSYAGSNNWVFGGGTKVTVLGQP
KAAPSVTLFPPSSEELQANKATLVCLISDFYPGAVTVAWKADSSPVKAGVETTTPSKQSNN
KYAASSYLSLTPEQWKSHRSYSCQVTHEGSTVEKTVAPTECGGGGSGGGGSGGGGSGG
GGSGGGGSGGGGGGGGSGGGGSGGGGSGGGGSGGGGSGGGGSGGGGSGGGGSE
VQLVQSGADVKRPGASVKVSCKISGDSFTAYFIHWVRQAPGQGLEWMGWFNPDSGTADS
AQKFHGRVTMTRDTSSSTAFLELSRLRSDDTAVYYCVRQHRGNTFAPWGRGTLVTVSSAS
TKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDGGGGSGGGGSGGGGSGGG
GSGGGGSGGSSQIRQNYSTDVEAAVNSLVNLYLQASYTYLSLGFYFDRDDVALEGVSHF
FRELAEEKREGYERLLKMQNQRGGRALFQDIKKPAEDEWGKTPDAMKAAMALEKKLNQ
ALLDLHALGSARTDPHLCDFLETHFLDEEVKLIKKMGDHLTNLHRLGGPEAGLGEYLFER
LTLRHD;
T1015A02-hFerr
(SEQ ID NO: 42)
AQAVLTQPSSASGTPGQRVTIPCSGSSSNIGGNTVNWYQQLPGTAPKLLIYGNDQR
PSGVPDRFSGSKSGTSASLAITGLQSEDEADYYCAAWDDSLIGYVFGTGTQLTVLGQPKAA
PSVTLFPPSSEELQANKATLVCLISDFYPGAVTVAWKADSSPVKAGVETTTPSKQSNNKYA
ASSYLSLTPEQWKSHRSYSCQVTHEGSTVEKTVAPTECGGGGSGGGGSGGGGSGGGGS
GGGGSGGGGSGGGGSGGGGSGGGGSGGGGSGGGGSGGGGSGGGGSGGGGSQVQL
QESGPGLVKPSQTLSLKCNVSGGSIGTGDYYWSWIRQPPGKGLEWIGYIHSSGSTYYKPSL
RSRLTVSMDTSRNQFSLKLTSVTAADTALYYCVREWANGDHWSAFDLWGQGTLVTVSSA
STKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDGGGGGGGGSGGGGSGG
GGSGGGGSGGMSSQIRQNYSTDVEAAVNSLVNLYLQASYTYLSLGFYFDRDDVALEGVS
HFFRELAEEKREGYERLLKMQNQRGGRALFQDIKKPAEDEWGKTPDAMKAAMALEKKL
NQALLDLHALGSARTDPHLCDFLETHFLDEEVKLIKKMGDHLTNLHRLGGPEAGLGEYLF
ERLTLRHD;
or
(SEQ ID NO: 43)
AQAVLTQPSSASGTPGQRVTIPCSGSSSNIGGNTVNWYQQLPGTAPKLLIYGNDQR
PSGVPDRFSGSKSGTSASLAITGLQSEDEADYYCAAWDDSLIGYVFGTGTQLTVLGQPKAA
PSVTLFPPSSEELQANKATLVCLISDFYPGAVTVAWKADSSPVKAGVETTTPSKQSNNKYA
ASSYLSLTPEQWKSHRSYSCQVTHEGSTVEKTVAPTECGGGGSGGGGSGGGGSGGGGS
GGGGSGGGGSGGGGSGGGGSGGGGSGGGGSGGGGSGGGGSGGGGSGGGGSQVQL
QESGPGLVKPSQTLSLKCNVSGGSIGTGDYYWSWIRQPPGKGLEWIGYIHSSGSTYYKPSL
RSRLTVSMDTSRNQFSLKLTSVTAADTALYYCVREWANGDHWSAFDLWGQGTLVTVSSA
STKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDGGGGSGGGGSGGGGSGG
GGSGGGGSGGSSQIRQNYSTDVEAAVNSLVNLYLQASYTYLSLGFYFDRDDVALEGVSH
FFRELAEEKREGYERLLKMQNQRGGRALFQDIKKPAEDEWGKTPDAMKAAMALEKKLN
QALLDLHALGSARTDPHLCDFLETHFLDEEVKLIKKMGDHLTNLHRLGGPEAGLGEYLFE
RLTLRHD;
T1015A07-hFerr
(SEQ ID NO: 44)
AQSALTQPASMSGSPGQSITISCTGTSSDVGGYNYVSWYQQHPGKAPKLMIYAVT
NRPSGVSNRFSASKSGNTASLTISGLQAEDEADYYCSSYTSSNTWVFGGGTKVTVLGQPK
AAPSVTLFPPSSEELQANKATLVCLISDFYPGAVTVAWKADSSPVKAGVETTTPSKQSNNK
YAASSYLSLTPEQWKSHRSYSCQVTHEGSTVEKTVAPTECGGGGSGGGGSGGGGSGGG
GSGGGGSGGGGSGGGGSGGGGSGGGGSGGGGSGGGGSGGGGSGGGGSGGGGSEV
QLAQSGAEVNKPGASVKVSCKISGDSFTAYFIHWLRQAPGEGLEWMGWFNPISGTADSPQ
KFHGRVAMTRDTSISTAYMELTRLASDDTAIYYCARQHHSNTFDPWGQGTLVTVSSASTKG
PSVFPLAPSSKSTSGGTAALGCLVKDYFPEPVTVSWNSGALTSGVHTFPAVLQSSGLYSLS
SWVTVPSSSLGTQTYICNVNHKPSNTKVDKKVEPKSCDGGGGSGGGGSGGGGSGGGGS
GGGGSGGMSSQIRQNYSTDVEAAVNSLVNLYLQASYTYLSLGFYFDRDDVALEGVSHFF
RELAEEKREGYERLLKMQNQRGGRALFQDIKKPAEDEWGKTPDAMKAAMALEKKLNQA
LLDLHALGSARTDPHLCDFLETHFLDEEVKLIKKMGDHLTNLHRLGGPEAGLGEYLFERL
TLRHD;
or
(SEQ ID NO: 45)
AQSALTQPASMSGSPGQSITISCTGTSSDVGGYNYVSWYQQHPGKAPKLMIYAVT
NRPSGVSNRFSASKSGNTASLTISGLQAEDEADYYCSSYTSSNTWVFGGGTKVTVLGQPK
AAPSVTLFPPSSEELQANKATLVCLISDFYPGAVTVAWKADSSPVKAGVETTTPSKQSNNK
YAASSYLSLTPEQWKSHRSYSCQVTHEGSTVEKTVAPTECGGGGSGGGGSGGGGSGGG
GSGGGGSGGGGSGGGGSGGGGSGGGGSGGGGSGGGGSGGGGSGGGGSGGGGSEV
QLAQSGAEVNKPGASVKVSCKISGDSFTAYFIHWLRQAPGEGLEWMGWFNPISGTADSPQ
KFHGRVAMTRDTSISTAYMELTRLASDDTAIYYCARQHHSNTFDPWGQGTLVTVSSASTKG
PSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDGGGGSGGGGSGGGGSGGGGS
GGGGSGGSSQIRQNYSTDVEAAVNSLVNLYLQASYTYLSLGFYFDRDDVALEGVSHFFRE
LAEEKREGYERLLKMQNQRGGRALFQDIKKPAEDEWGKTPDAMKAAMALEKKLNQALL
DLHALGSARTDPHLCDFLETHFLDEEVKLIKKMGDHLTNLHRLGGPEAGLGEYLFERLTL
RHD;
T1006F07-hFerr
(SEQ ID NO: 46)
AQSVLTQPPSVSVSPGQAARITCSGDKLGDKYASWYQQRPGQSPVLVIYQDNKRP
SGIPERFSGSNSGNTATLKISGTQAMDEADYYCLAWDSSADWVFGGGTKVTVLGQPKAAP
SVTLFPPSSEELQANKATLVCLISDFYPGAVTVAWKADSSPVKAGVETTTPSKQSNNKYAA
SSYLSLTPEQWKSHRSYSCQVTHEGSTVEKTVAPTECGGGGSGGGGSGGGGGGGGSG
GGGSGGGGSGGGGSGGGGSGGGGSGGGGSGGGGSGGGGSGGGGSGGGGSEVQLLE
SGGGLVQPGGSLRLSCAASGFTFSSYAMSWVRQAPGKGLEWVSAISGSGGSTYYADSVK
GRFTISRDNSKNTLYLQMNSLRAEDTAVYYCAREPSFQQWGHYSYGMDVWGQGTMVTVS
SASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDGGGGSGGGGSGGGGS
GGGGSGGGGSGGMSSQIRQNYSTDVEAAVNSLVNLYLQASYTYLSLGFYFDRDDVALEG
VSHFFRELAEEKREGYERLLKMQNQRGGRALFQDIKKPAEDEWGKTPDAMKAAMALEK
KLNQALLDLHALGSARTDPHLCDFLETHFLDEEVKLIKKMGDHLTNLHRLGGPEAGLGEY
LFERLTLRHD;
or
(SEQ ID NO: 47)
AQSVLTQPPSVSVSPGQAARITCSGDKLGDKYASWYQQRPGQSPVLVIYQDNKRP
SGIPERFSGSNSGNTATLKISGTQAMDEADYYCLAWDSSADWVFGGGTKVTVLGQPKAAP
SVTLFPPSSEELQANKATLVCLISDFYPGAVTVAWKADSSPVKAGVETTTPSKQSNNKYAA
SSYLSLTPEQWKSHRSYSCQVTHEGSTVEKTVAPTECGGGGSGGGGSGGGGSGGGGSG
GGGSGGGGSGGGGSGGGGSGGGGGGGGSGGGGSGGGGSGGGGSGGGGSEVQLLE
SGGGLVQPGGSLRLSCAASGFTFSSYAMSWVRQAPGKGLEWVSAISGSGGSTYYADSVK
GRFTISRDNSKNTLYLQMNSLRAEDTAVYYCAREPSFQQWGHYSYGMDVWGQGTMVTVS
SASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDGGGGSGGGGSGGGGS
GGGGSGGGGSGGSSQIRQNYSTDVEAAVNSLVNLYLQASYTYLSLGFYFDRDDVALEGV
SHFFRELAEEKREGYERLLKMQNQRGGRALFQDIKKPAEDEWGKTPDAMKAAMALEKK
LNQALLDLHALGSARTDPHLCDFLETHFLDEEVKLIKKMGDHLTNLHRLGGPEAGLGEYL
FERLTLRHD;
42/43-hFerr
(SEQ ID NO: 48)
LEDIQMIQSPLSLPVIPGEPASMSCRSSRSLLHSNGNNYLQWYLQKPGQSPQLLIYL
GSNRASGVPDRFSGSGSGTDFTLKISRVEAEDVGIYYCMQGLQLPTTFGGTKVIKRTVAAP
SVFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGECGGGGSGGGGSGGGGSGGG
GSGGGGSGGGGSGGGGSGGGGSGGGGSGGGGSGGGGSGGGGSGGGGSGGGGSQV
QLVQSGAEVKKPGASVKVSCKASGYTFTNYDINWVRQAPGQGLEWMGISAYTGNTNYAQ
KLQGRVTMTTDTSTSTAYMELRSLRSDDTAVYYCVRDYHDSNGYYYFDYWGQGTLVTVSA
STKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDGGGGSGGGGSGGGGSGG
GGSGGGGSGGMSSQIRQNYSTDVEAAVNSLVNLYLQASYTYLSLGFYFDRDDVALEGVS
HFFRELAEEKREGYERLLKMQNQRGGRALFQDIKKPAEDEWGKTPDAMKAAMALEKKL
NQALLDLHALGSARTDPHLCDFLETHFLDEEVKLIKKMGDHLTNLHRLGGPEAGLGEYLF
ERLTLRHD;
or
(SEQ ID NO: 49)
LEDIQMIQSPLSLPVIPGEPASMSCRSSRSLLHSNGNNYLQWYLQKPGQSPQLLIYL
GSNRASGVPDRFSGSGSGTDFTLKISRVEAEDVGIYYCMQGLQLPTTFGGTKVIKRTVAAP
SVFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGECGGGGSGGGGSGGGGSGGG
GSGGGGSGGGGSGGGGSGGGGSGGGGSGGGGSGGGGSGGGGSGGGGSGGGGSQV
QLVQSGAEVKKPGASVKVSCKASGYTFTNYDINWVRQAPGQGLEWMGISAYTGNTNYAQ
KLQGRVTMTTDTSTSTAYMELRSLRSDDTAVYYCVRDYHDSNGYYYFDYWGQGTLVTVSA
STKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDGGGGSGGGGSGGGGSGG
GGSGGGGSGGSSQIRQNYSTDVEAAVNSLVNLYLQASYTYLSLGFYFDRDDVALEGVSH
FFRELAEEKREGYERLLKMQNQRGGRALFQDIKKPAEDEWGKTPDAMKAAMALEKKLN
QALLDLHALGSARTDPHLCDFLETHFLDEEVKLIKKMGDHLTNLHRLGGPEAGLGEYLFE
RLTLRHD;
44/45-hFerr
(SEQ ID NO: 50)
LEEIVLTQSPFFQSVTPKEKVTITCRASQNIGSSLHWYQQKPDQSPKLLIKSASQSFS
GVPSRFSGSGSGTDFTLTINSLEAEDAATYYCHQSSSLPFTFGPGTKVDIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGECGGGGSGGGGGGGGSGGGGSGG
GGSGGGGSGGGGSGGGGSGGGGSGGGGSGGGGSGGGGSGGGGSGGGGSQVQLVQ
SGGGVVQPGRSLRLSCAASGFTFRTYGMHWVRQAPGKGLEWVAVLWYDGTNKYYADSV
KGRFAISRDNSNNTLYLQMNSLRAEDAAVYYCARDGSYYYDSSGYYYVGGFDYWGQGTL
VTVSSASTKGPSVFPLAPSSKSTSGGTAALGCLVKDYFPEPVTVSWNSGALTSGVHTFPAV
LQSSGLYSLSSVTVPSSSLGTQTYICNVNHKPSNTKVDKKVEPKSCDGGGGSGGGGSGG
GGSGGGGSGGGGSGGMSSQIRQNYSTDVEAAVNSLVNLYLQASYTYLSLGFYFDRDDV
ALEGVSHFFRELAEEKREGYERLLKMQNQRGGRALFQDIKKPAEDEWGKTPDAMKAAM
ALEKKLNQALLDLHALGSARTDPHLCDFLETHFLDEEVKLIKKMGDHLTNLHRLGGPEAG
LGEYLFERLTLRHD;
or
(SEQ ID NO: 51)
LEEIVLTQSPFFQSVTPKEKVTITCRASQNIGSSLHWYQQKPDQSPKLLIKSASQSFS
GVPSRFSGSGSGTDFTLTINSLEAEDAATYYCHQSSSLPFTFGPGTKVDIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGECGGGGSGGGGSGGGGSGGGGSGG
GGSGGGGSGGGGSGGGGSGGGGSGGGGSGGGGSGGGGSGGGGSGGGGSQVQLVQ
SGGGWQPGRSLRLSCAASGFTFRTYGMHWVRQAPGKGLEWVAVLWYDGTNKYYADSV
KGRFAISRDNSNNTLYLQMNSLRAEDAAVYYCARDGSYYYDSSGYYYVGGFDYWGQGTL
VTVSSASTKGPSVFPLAPSSKSTSGGTAALGCLVKDYFPEPVTVSWNSGALTSGVHTFPAV
LQSSGLYSLSSWTVPSSSLGTQTYICNVNHKPSNTKVDKKVEPKSCDGGGGSGGGGSGG
GGSGGGGSGGGGSGGSSQIRQNYSTDVEAAVNSLVNLYLQASYTYLSLGFYFDRDDVAL
EGVSHFFRELAEEKREGYERLLKMQNQRGGRALFQDIKKPAEDEWGKTPDAMKAAMAL
EKKLNQALLDLHALGSARTDPHLCDFLETHFLDEEVKLIKKMGDHLTNLHRLGGPEAGLG
EYLFERLTLRHD;
46/47-hFerr
(SEQ ID NO: 52)
LEEVVLTQSPGTLSLSLGERATLSCRASQSVSSYLAWYQHKPGQAPRLLIYGTSSR
ATGIPDRFSGSGSGTNFTLTISRLEPEDFAVYYCQQYGSLPFTFGPGTKVDIKRTVAAPSVFI
FPPSDEQLKSGTASVVCLLNNFYPREAKVQWKVDNALQSGNSQESVTEQDSKDSTYSLSS
TLTLSKADYEKHKVYACEVTHQGLSSPVTKSFNRGECGGGGSGGGGSGGGGSGGGGSG
GGGSGGGGSGGGGSGGGGSGGGGSGGGGSGGGGSGGGGSGGGGSGGGGSQVQLV
ESGGGVVQPGRSLRLSCSASGFTFSSGIHWVRQAPGKGLEWVVVMWYAGSNEYYADSV
KGRFTISRDNSKNTLYLQMNSLRAEDTAVYYCARDQGVLLRFGELRGYYGMDVWGQGTT
VTVSSASTKGPSVFPLAPSSKSTSGGTAALGCLVKDYFPEPVTVSWNSGALTSGVHTFPAV
LQSSGLYSLSSWTVPSSSLGTQTYICNVNHKPSNTKVDKKVEPKSCDGGGGSGGGGSGG
GGSGGGGSGGGGSGGMSSQIRQNYSTDVEAAVNSLVNLYLQASYTYLSLGFYFDRDDV
ALEGVSHFFRELAEEKREGYERLLKMQNQRGGRALFQDIKKPAEDEWGKTPDAMKAAM
ALEKKLNQALLDLHALGSARTDPHLCDFLETHFLDEEVKLIKKMGDHLTNLHRLGGPEAG
LGEYLFERLTLRHD;
or
(SEQ ID NO: 53)
LEEVVLTQSPGTLSLSLGERATLSCRASQSVSSYLAWYQHKPGQAPRLLIYGTSSR
ATGIPDRFSGSGSGTNFTLTISRLEPEDFAVYYCQQYGSLPFTFGPGTKVDIKRTVAAPSVFI
FPPSDEQLKSGTASVVCLLNNFYPREAKVQWKVDNALQSGNSQESVTEQDSKDSTYSLSS
TLTLSKADYEKHKVYACEVTHQGLSSPVTKSFNRGECGGGGSGGGGSGGGGSGGGGSG
GGGSGGGGSGGGGSGGGGSGGGGSGGGGSGGGGGGGGSGGGGSGGGGSQVQLV
ESGGGVVQPGRSLRLSCSASGFTFSSGIHWVRQAPGKGLEWVVVMWYAGSNEYYADSV
KGRFTISRDNSKNTLYLQMNSLRAEDTAVYYCARDQGVLLRFGELRGYYGMDVWGQGTT
VTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDGGGGSGGGGSGG
GGSGGGGSGGGGSGGSSQIRQNYSTDVEAAVNSLVNLYLQASYTYLSLGFYFDRDDVAL
EGVSHFFRELAEEKREGYERLLKMQNQRGGRALFQDIKKPAEDEWGKTPDAMKAAMAL
EKKLNQALLDLHALGSARTDPHLCDFLETHFLDEEVKLIKKMGDHLTNLHRLGGPEAGLG
EYLFERLTLRHD;
Fc-hFerr LALAP 1253A
(SEQ ID NO: 54)
DKTHTCPPCPAPEAAGGPSVFLFPPKPKDTLMASRTPEVTCVVVDVSHEDPEVKFN
WYVDGVEVHNAKTKPREEQYNSTYRVVSVLTVLHQDWLNGKEYKCKVSNKALGAPIEKTIS
KAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGKGGGGSGGGG
SGGGGSGGGGSGGGGSGGGGSGGGGSGGGGSGGGGSGGGGSGGGGSGGGGSGGG
GSGGGGSDKTHTCPPCPAPEAAGGPSVFLFPPKPKDTLMASRTPEVTCVVVDVSHEDPEV
KFNWYVDGVEVHNAKTKPREEQYNSTYRVVSVLTVLHQDWLNGKEYKCKVSNKALGAPIE
KTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGKGGGGSG
GGGSGGGGSGGGGSGGGGSGGMSSQIRQNYSTDVEAAVNSLVNLYLQASYTYLSLGFY
FDRDDVALEGVSHFFRELAEEKREGYERLLKMQNQRGGRALFQDIKKPAEDEWGKTPD
AMKAAMALEKKLNQALLDLHALGSARTDPHLCDFLETHFLDEEVKLIKKMGDHLTNLHRL
GGPEAGLGEYLFERLTLRHD;
or
(SEQ ID NO: 55)
DKTHTCPPCPAPEAAGGPSVFLFPPKPKDTLMASRTPEVTCVVVDVSHEDPEVKFN
WYVDGVEVHNAKTKPREEQYNSTYRVVSVLTVLHQDWLNGKEYKCKVSNKALGAPIEKTIS
KAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGKGGGGSGGGG
SGGGGSGGGGSGGGGSGGGGSGGGGSGGGGSGGGGSGGGGSGGGGSGGGGSGGG
GSGGGGSDKTHTCPPCPAPEAAGGPSVFLFPPKPKDTLMASRTPEVTCVVVDVSHEDPEV
KFNWYVDGVEVHNAKTKPREEQYNSTYRVVSVLTVLHQDWLNGKEYKCKVSNKALGAPIE
KTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGKGGGGSG
GGGSGGGGSGGGGSGGGGSGGSSQIRQNYSTDVEAAVNSLVNLYLQASYTYLSLGFYF
DRDDVALEGVSHFFRELAEEKREGYERLLKMQNQRGGRALFQDIKKPAEDEWGKTPDA
MKAAMALEKKLNQALLDLHALGSARTDPHLCDFLETHFLDEEVKLIKKMGDHLTNLHRL
GGPEAGLGEYLFERLTLRHD;
hFerr
(SEQ ID NO: 56)
MSSQIRQNYSTDVEAAVNSLVNLYLQASYTYLSLGFYFDRDDVALEGVSHFFREL
AEEKREGYERLLKMQNQRGGRALFQDIKKPAEDEWGKTPDAMKAAMALEKKLNQALLD
LHALGSARTDPHLCDFLETHFLDEEVKLIKKMGDHLTNLHRLGGPEAGLGEYLFERLTLR
HD;
or
(SEQ ID NO: 57)
SSQIRQNYSTDVEAAVNSLVNLYLQASYTYLSLGFYFDRDDVALEGVSHFFRELAE
EKREGYERLLKMQNQRGGRALFQDIKKPAEDEWGKTPDAMKAAMALEKKLNQALLDLH
ALGSARTDPHLCDFLETHFLDEEVKLIKKMGDHLTNLHRLGGPEAGLGEYLFERLTLRHD.

Also described herein are compositions comprising the nanocage, such as therapeutic or prophylactic compositions. Related methods and uses for treating and/or preventing cancer are also described, wherein the method or use comprises administering the nanocage or composition described herein to a subject in need thereof.

In aspects, the nanocage is capable of killing DR4- and/or DR-5-positive cancer cells with an IC₅₀ value of less than about 0.1 μg/ml, less than about 0.01 μg/ml, or less than about 0.001 μg/ml, as determined in an in vitro cell killing assay. In aspects, the nanocage is capable of killing DR4- and/or DR-5-positive cancer cells with an IC₅₀ value of less than about 10 pM, less than about 5 pM, less than about 2 pM, less than about 1 pM, less than about 0.5 pM, less than about 0.4 pM, less than about 0.35 pM, less than about 0.25 pM, les than about 0.2 pM, less than about 0.15 pM, less than about 0.1 pM, or less than about 0.05 pM, as determined in an in vitro cell killing assay.

In additional or alternative aspects, the nanocage is capable of killing DR4- and/or DR5-positive cancer cells with an IC₅₀ value that is at least about 10-fold, at least about 100-fold, at least about 1000-fold, at least about 2,000-fold, at least about 5,000-fold, at least about 10,000-fold, at least about 15,000-fold, at least about 20,000-fold, at least about 50,000-fold, at least about 75,000-fold, at least about 100,000-fold, at least about 150,000-fold, at least about 200,000-fold, at least about 250,000-fold, at least about 300,00-fold, or at least about 400,000-fold lower than the IC₅₀ value for a reference molecule, e.g., a corresponding IgG. In other words, the nanocage is at least about 10, at least about 100, at least about 1000, at least about 2,000, at least about 5,000, at least about 10,000, at least about 15,000, at least about 20,000, at least about 50,000, at least about 75,000, at least about 100,000, at least about 150,000, at least about 200,000, at least about 250,000, or at least about 400,000 times more potent than the reference molecule, e.g., the corresponding IgG on a mass and/or molar basis.

It will be understood that polypeptides substantially identical to those described herein are also contemplated. A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G).

“Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 85% identical; in another example, the substantially identical sequences may be at least 70, 75, 80, 85, 90, 95, 96, 97, 98, 99, or 100% (or any percentage there between) identical at the amino acid level to sequences described herein. In specific aspects, the substantially identical sequences retain the activity and specificity of the reference sequence. In a non-limiting embodiment, the difference in sequence identity may be due to conservative amino acid mutation(s).

The polypeptides or fusion proteins of the present invention may also comprise additional sequences to aid in their expression, detection or purification. Any such sequences or tags known to those of skill in the art may be used. For example, and without wishing to be limiting, the fusion proteins may comprise a targeting or signal sequence (for example, but not limited to ompA), a detection tag, exemplary tag cassettes include Strep tag, or any variant thereof; see, e.g., U.S. Pat. No. 7,981,632, His tag, Flag tag having the sequence motif DYKDDDDK, Xpress tag, Avi tag, Calmodulin tag, Polyglutamate tag, HA tag, Myc tag, Nus tag, S tag, SBP tag, Softag 1, Softag 3, V5 tag, CREB-binding protein (CBP), glutathione S-transferase (GST), maltose binding protein (MBP), green fluorescent protein (GFP), Thioredoxin tag, or any combination thereof; a purification tag (for example, but not limited to a His₅ or His₆), or a combination thereof.

In another example, the additional sequence may be a biotin recognition site such as that described by Cronan et al in WO 95/04069 or Voges et al in WO/2004/076670. As is also known to those of skill in the art, linker sequences may be used in conjunction with the additional sequences or tags.

More specifically, a tag cassette may comprise an extracellular component that can specifically bind to an antibody with high affinity or avidity. Within a single chain fusion protein structure, a tag cassette may be located (a) immediately amino-terminal to a connector region, (b) interposed between and connecting linker modules, (c) immediately carboxy-terminal to a binding domain, (d) interposed between and connecting a binding domain (e.g., scFv or scFab) to an effector domain, (e) interposed between and connecting subunits of a binding domain, or (f) at the amino-terminus of a single chain fusion protein. In certain embodiments, one or more junction amino acids may be disposed between and connecting a tag cassette with a hydrophobic portion, or disposed between and connecting a tag cassette with a connector region, or disposed between and connecting a tag cassette with a linker module, or disposed between and connecting a tag cassette with a binding domain.

Also encompassed herein are isolated or purified fusion proteins, polypeptides, or fragments thereof immobilized onto a surface using various methodologies; for example, and without wishing to be limiting, the polypeptides may be linked or coupled to the surface via His-tag coupling, biotin binding, covalent binding, adsorption, and the like. The solid surface may be any suitable surface, for example, but not limited to the well surface of a microtiter plate, channels of surface plasmon resonance (SPR) sensor chips, membranes, beads (such as magnetic-based or sepharose-based beads or other chromatography resin), glass, a film, or any other useful surface.

In other aspects, the fusion proteins may be linked to a cargo molecule; the fusion proteins may deliver the cargo molecule to a desired site and may be linked to the cargo molecule using any method known in the art (recombinant technology, chemical conjugation, chelation, etc.). The cargo molecule may be any type of molecule, such as a therapeutic or diagnostic agent.

In some aspects, the cargo molecule is a protein and is fused to the fusion protein such that the cargo molecule is contained in the nanocage internally. In other aspects, the cargo molecule is not fused to the fusion protein and is contained in the nanocage internally. The cargo molecule is typically a protein, a small molecule, a radioisotope, or a magnetic particle.

The fusion proteins described herein specifically bind to their targets. Antibody specificity, which refers to selective recognition of an antibody for a particular epitope of an antigen, of the antibodies or fragments described herein can be determined based on affinity and/or avidity. Affinity, represented by the equilibrium constant for the dissociation of an antigen with an antibody (K_{D}), measures the binding strength between an antigenic determinant (epitope) and an antibody binding site. Avidity is the measure of the strength of binding between an antibody with its antigen. Antibodies typically bind with a K_{D} of 10⁻⁵ to 10⁻¹¹ M. Any K_{D} greater than 10⁻⁴ M is generally considered to indicate non-specific binding. The lesser the value of the K_{D}, the stronger the binding strength between an antigenic determinant and the antibody binding site. In aspects, the antibodies described herein have a K_{D} of less than 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, or 10⁻¹⁵ M.

Also described herein are nucleic acid molecules encoding the fusion proteins and polypeptides described herein, as well as vectors comprising the nucleic acid molecules and host cells comprising the vectors.

Polynucleotides encoding the fusion proteins described herein include polynucleotides with nucleic acid sequences that are substantially the same as the nucleic acid sequences of the polynucleotides of the present invention. “Substantially the same” nucleic acid sequence is defined herein as a sequence with at least 70%, at least 75%, at least 80%, at least 85%, at least 90%, at least 91%, at least 92%, at least 93%, at least 94%, at least 95% identity to another nucleic acid sequence when the two sequences are optimally aligned (with appropriate nucleotide insertions or deletions) and compared to determine exact matches of nucleotides between the two sequences.

Suitable sources of polynucleotides that encode fragments of antibodies include any cell, such as hybridomas and spleen cells, that express the full-length antibody. The fragments may be used by themselves as antibody equivalents, or may be recombined into equivalents, as described above. The DNA deletions and recombinations described in this section may be carried out by known methods, such as those described in the published patent applications listed above in the section entitled “Functional Equivalents of Antibodies” and/or other standard recombinant DNA techniques, such as those described below. Another source of DNAs are single chain antibodies produced from a phage display library, as is known in the art.

Additionally, expression vectors are provided containing the polynucleotide sequences previously described operably linked to an expression sequence, a promoter and an enhancer sequence. A variety of expression vectors for the efficient synthesis of antibody polypeptide in prokaryotic, such as bacteria and eukaryotic systems, including but not limited to yeast and mammalian cell culture systems have been developed. The vectors of the present invention can comprise segments of chromosomal, non-chromosomal and synthetic DNA sequences.

Any suitable expression vector can be used. For example, prokaryotic cloning vectors include plasmids from E. coli, such as colEI, pCRI, pBR322, pMB9, pUC, pKSM, and RP4. Prokaryotic vectors also include derivatives of phage DNA such as MI3 and other filamentous single-stranded DNA phages. An example of a vector useful in yeast is the 2 μ plasmid. Suitable vectors for expression in mammalian cells include well-known derivatives of SV-40, adenovirus, retrovirus-derived DNA sequences and shuttle vectors derived from combination of functional mammalian vectors, such as those described above, and functional plasmids and phage DNA.

Additional eukaryotic expression vectors are known in the art (e.g., P J. Southern & P. Berg, J. Mol. Appl. Genet, 1:327-341 (1982); Subramani et al, Mol. Cell. Biol, 1: 854-864 (1981); Kaufman & Sharp, “Amplification And Expression of Sequences Cotransfected with a Modular Dihydrofolate Reductase Complementary DNA Gene,” J. Mol. Biol, 159:601-621 (1982); Kaufman & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), all of which are incorporated by reference herein).

The expression vectors typically contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Also described herein are recombinant host cells containing the expression vectors previously described. The fusion proteins described herein can be expressed in cell lines other than in hybridomas. Nucleic acids, which comprise a sequence encoding a polypeptide according to the invention, can be used for transformation of a suitable mammalian host cell.

Cell lines of particular preference are selected based on high level of expression, constitutive expression of protein of interest and minimal contamination from host proteins. Mammalian cell lines available as hosts for expression are well known in the art and include many immortalized cell lines, such as but not limited to, HEK 293 cells, Chinese Hamster Ovary (CHO) cells, Baby Hamster Kidney (BHK) cells and many others. Suitable additional eukaryotic cells include yeast and other fungi. Useful prokaryotic hosts include, for example, E. coli, such as E. coli SG-936, E. coli HB 101, E. coli W3110, E. coli X1776, E. coli X2282, E. coli DHI, and E. coli MRC1, E. coli T7 shuffle, Pseudomonas, Bacillus, such as Bacillus subtilis, and Streptomyces.

These present recombinant host cells can be used to produce fusion proteins by culturing the cells under conditions permitting expression of the polypeptide and purifying the polypeptide from the host cell or medium surrounding the host cell. Targeting of the expressed polypeptide for secretion in the recombinant host cells can be facilitated by inserting a signal or secretory leader peptide-encoding sequence (See, Shokri et al, (2003) Appl Microbiol Biotechnol. 60(6): 654-664, Nielsen et al, Prot. Eng., 10:1-6 (1997); von Heinje et al., Nucl. Acids Res., 14:4683-4690 (1986), all of which are incorporated by reference herein) at the 5′ end of the antibody-encoding gene of interest. These secretory leader peptide elements can be derived from either prokaryotic or eukaryotic sequences. Accordingly suitably, secretory leader peptides are used, being amino acids joined to the N-terminal end of a polypeptide to direct movement of the polypeptide out of the host cell cytosol and secretion into the medium.

The fusion proteins described herein can be fused to additional amino acid residues. Such amino acid residues can be a peptide tag to facilitate isolation, for example. Other amino acid residues for homing of the antibodies to specific organs or tissues are also contemplated.

It will be understood that a Fab-nanocage can be generated, e.g., by co-transfection of plasmids, one encoding a fusion protein comprising an Fab heavy chain fused to a ferritin chain (e.g., ferritin light chain), and another encoding an Fab light chain. Alternatively, single-chain Fab-ferritin nanocages can be used that only require transfection of one plasmid (e.g., using a plasmid that encodes a fusion protein comprising Fab light chain, Fab heavy chain, and a ferritin chain (e.g., ferritin light chain)). This can be done with linkers of different lengths between the Fab light chain and the Fab heavy chain for example 60 or 70 amino acids. When single-chain Fabs are used, it can be ensured that the heavy chain and light chain are paired. Tags (e.g. Flag, HA, myc, His6x, Strep, etc.) can also be added at the N terminus of the construct or within the linker for ease of purification as described above. Further, a tag system can be used to make sure many different Fabs are present on the same nanoparticle using serial/additive affinity chromatography steps when different Fab-nanoparticle plasmids are co-transfected. This provides multi-specificity to the nanoparticles. Protease sites (e.g. TEV, 3C, etc.) can be inserted to cleave linkers and tags after expression and/or purification, if desired.

Any suitable method or route can be used to administer the fusion proteins described herein. Routes of administration include, for example, oral, intravenous, intraperitoneal, subcutaneous, or intramuscular administration.

It is understood that the fusion proteins described herein, where used in a mammal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal.

Although the fusion peptides and Multabodies described herein are particularly useful for administration to humans, they may be administered to other mammals as well. The term “mammal” as used herein is intended to include, but is not limited to, humans, laboratory animals, domestic pets and farm animals.

The above disclosure generally describes the present invention. A more complete understanding can be obtained by reference to the following specific examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

Without further description, it is believed that one of ordinary skill in the art can, using the preceding description and the following illustrative examples, make and utilize the compounds of the present invention and practice the claimed methods. The following working examples therefore, specifically point out the typical aspects of the present invention, and are not to be construed as limiting in any way the remainder of the disclosure.

### EXAMPLES

### Example 1: Antibody Multimerization Enhances the Cytotoxic Capacity of the DR5 Conatumumab and Tigatuzumab Antibodies against Different Tumor Cells Reaching IC₅₀ Values in the ng/ml (pM) Range

### Materials and Methods

### Protein Expression and Purification of Multabodies (MB)

All genes were synthesized and cloned by GeneArt (Life Technologies) into the pcDNA3.4 expression vector. Multabodies were expressed transiently in ExpiCHO-S cells (Thermo Fisher Scientific) at a density of 6×10⁶ cells/mL with 60 μg of DNA per 100 ml of cells using ExpiFectamine CHO (Thermo Fisher Scientific) in a 4:1:1 ratio (scFab-human light chain apoferritin: scFc-humab light chain apoferritin: human light chain apoferritin). In the case of split ferritin MB the genes encoding scFab and scFc fragments linked to half apoferritin were generated by deletion of residues 1 to 90 (C-Ferritin) and 91 to 175 (N-Ferritin) of the light chain of human apoferritin.

Transient transfection of the split MB in ExpiCHO-S cells (Thermo Fisher Scientific) cells were obtained by mixing 67.5 μg of the plasmids scFab-human light chain apoferritin:scFc-human C-human light chain apoferritin:scFab-N-human light chain apoferritin in a 2:1:1 ratio. The DNA mixture was filtered and incubated at RT with 67.5 μl of ExpiFectamine CHO (Thermo Fisher Scientific) before adding to the cell culture. One day after transfection, 24 ml of ExpiCHO Feed and 0.6 ml of ExpiFectamine Enhancer were added to the cells and cultured for 7 additional days at 125 rpm oscillation at 37° C, 8% CO₂, and 80% humidity in a Multitron Pro shaker (Infors HT). ExpiCHO expression medium (Thermo Fisher Scientific) and vented non-baffled erlenmeyer shake flasks (Corning) were used. Cell suspensions were harvested by centrifugation at 5000×g for 15 min and supernatants were filtered through a 0.22 μm Steritop filter (EMD Millipore).

IgGs were transiently expressed by co-transfecting 90 μg of the LC and the HC in a 1:2 ratio and purified using HiTrap Protein A HP column (GE Healthcare) with 100 mM glycine pH 2.2 as the elution buffer. Eluted fractions were immediately neutralized with 1 M Tris-HCl, pH 9.0 and further purified using a Superdex 200 Increase size exclusion column (GE Healthcare). Multabodies were purified by affinity chromatography using a HiTrap Protein A HP column (GE Healthcare) with 3M MgCl₂ 10% glycerol and the eluted fraction was loaded onto a Superose 6 10/300 GL size exclusion column (GE Healthcare) in 20 mM sodium phosphate pH 8.0, 150 mM NaCl.

### Cell Viability Assay

NCI-H2122, NIC-H2228 and Colo205 11 cell lines were grown in RPMI 1640 media (Sigma) supplemented with 10% fetal bovine serum. MBA-MB-231, HT29 and HT15 cell lines were grown in DMEM media (Gibco, Invitrogen) supplemented with 10% fetal bovine serum. SW948 cell lines was grown in Leibovitz's L-15 Medium supplemented with 10% fetal bovine serum (100% Air). Capan-1 cell line was grown in Iscove's Modified Dulbecco's Medium supplemented with 20% fetal bovine serum.

5,000 cells/well of each cancer cell line in 100 μl media was co-cultured with 100 μl of 10-fold serial dilutions of the Multabody or the IgG at 37° C. After 24 h incubation, cell viability was monitored by adding 50 μl of CellTiter-Glo 2.0 reagent (Promega) to 200 μl of media containing cells. After 10 min incubation, 100 μl was transferred to a 96-well black plate (Sigma-Aldrich) to measure luminescence in relative light units (RLUs) using a Synergy Neo2 Multi-Mode Assay Microplate Reader (Biotek Instruments).

### Results

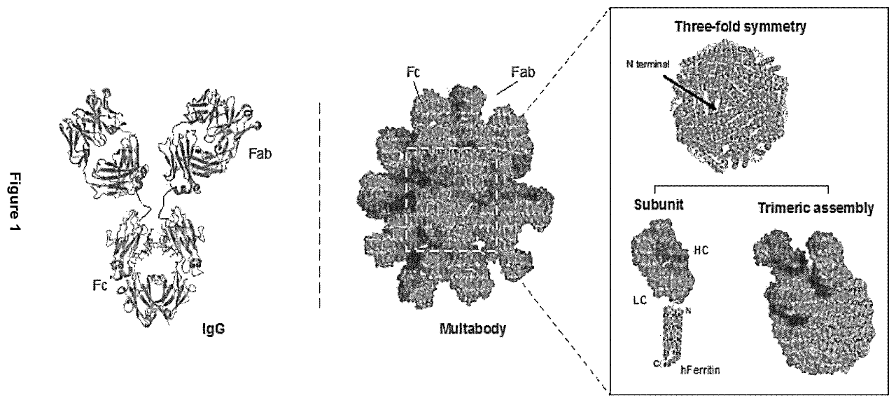

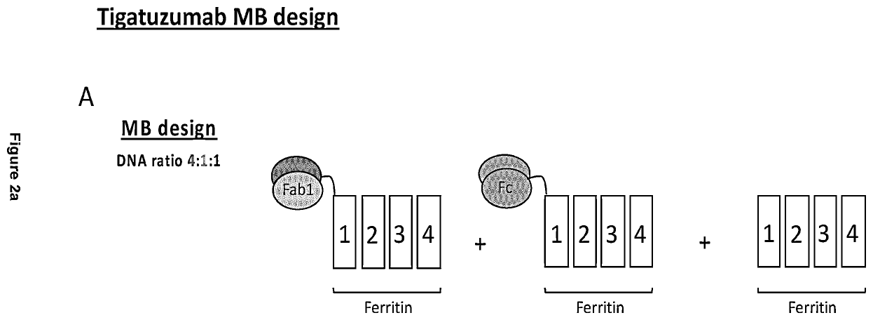

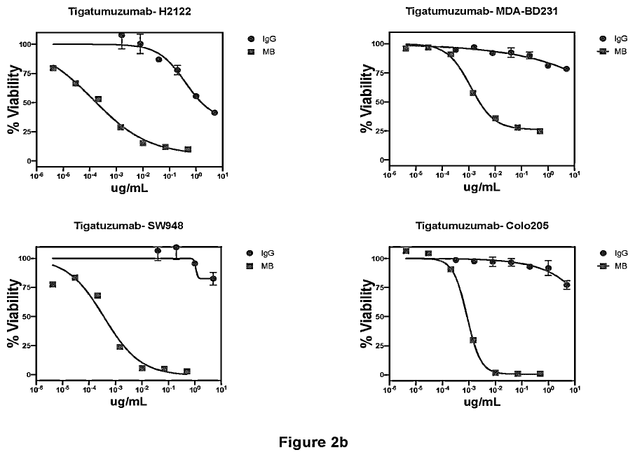

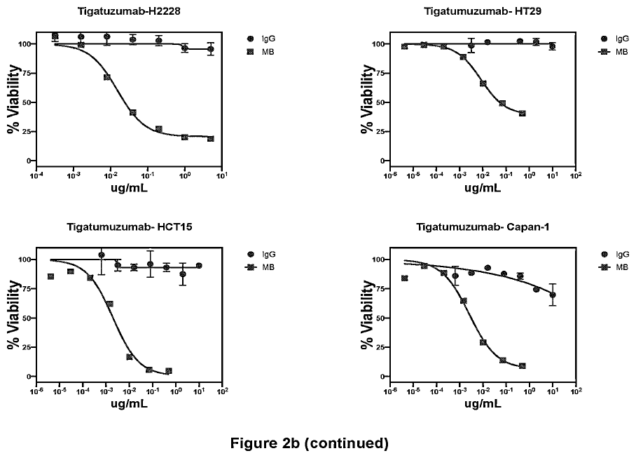

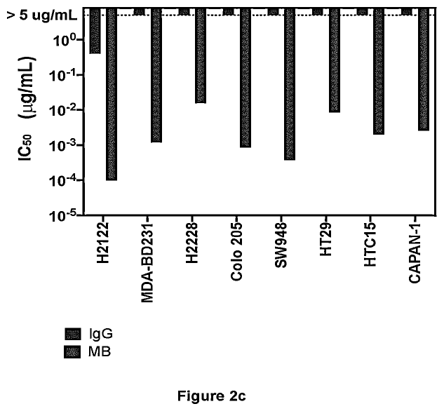

As shown in FIG. 1, the Multabody assembly displays Fabs to target trimeric receptors. Schematic representation showing Multabody valency (right) in comparison to a conventional Conatumumab IgG (left). Close-up view of Fab (dark red for the heavy chain and light red for light chain) clustering at the three-fold symmetry axes (light teal) of ferritin. Fragments in gold represent Fc fragments. Tigatuzumab MBs were generated by DNA cotransfection in a 4:1:1 ratio of the following components:scFab-human light chain apoferritin:scFc-humab light chain apoferritin:humab light chain apoferritin. As exemplified in FIG. 2A, both the scFab and the scFc were fused to the N-terminus of the full human light chain apoferritin.

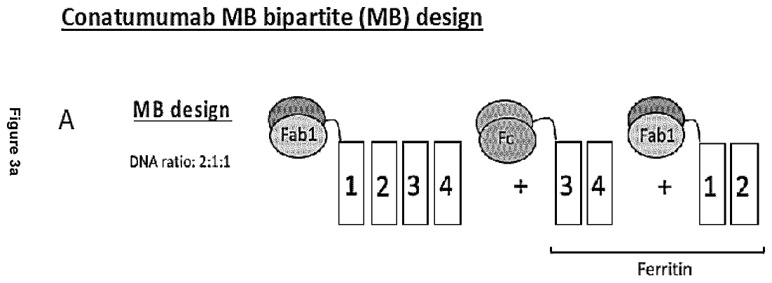

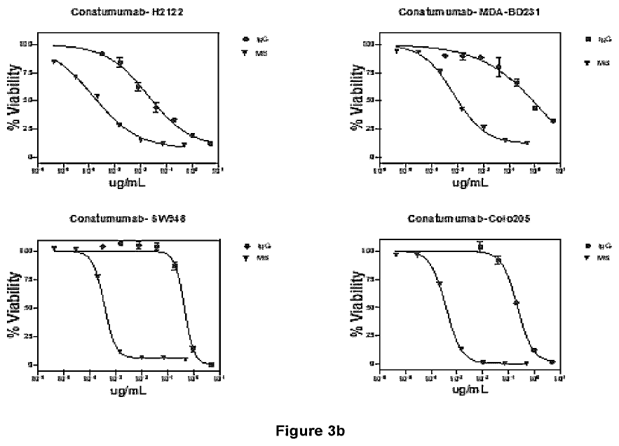

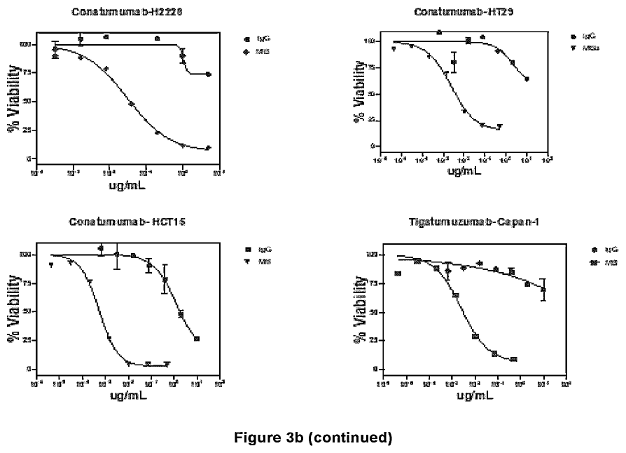

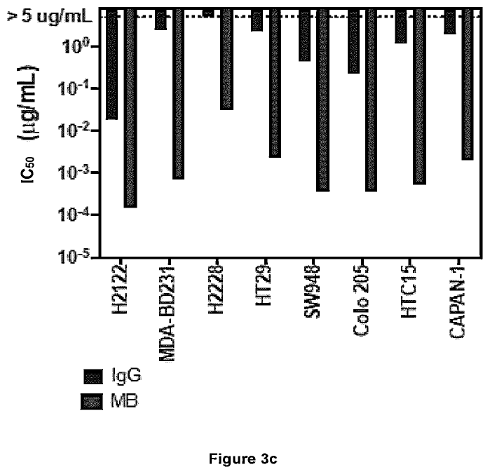

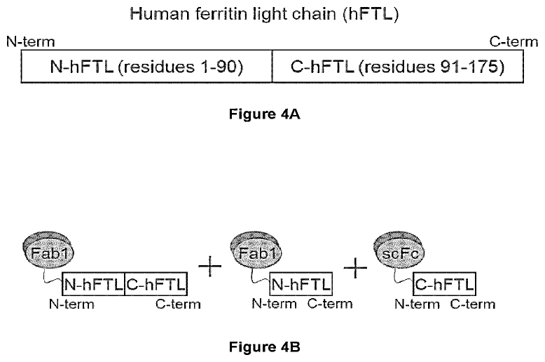

Conatumumab MBs were generated by DNA cotransfection in a 2:1:1 ratio of the following components:scFab-human light chain apoferritin:scFc-human C-human light chain apoferritin:scFab-N-human light chain apoferritin. As exemplified in FIG. 3A, the scFab was fused to the N-terminus of the full and the N half of the human light chain apoferritin, and the scFc was fused to the N-terminus of the C half of the humab light chain apoferritin. This design ensured that scFabs would consistently surround the three-fold axis of the self-assembled apoferritin, and thus optimally engage trimeric receptors.

As shown in FIGS. 2, 3 and Table 1, avidity enhances cell death against multiple cancer cell lines. Relative killing capacity of multiple cancer cell lines by Conatumumab and Tigatuzumab in the Multabody format vs the parental IgGs is summarized in Table 1. Notably, the majority of the cancer cells tested were resistant to Tigatuzumab IgG killing (IC₅₀ values>10 μg/mL tested). However, when the Fab region of Tigatuzumab was multimerized in the MB, IC₅₀ values as low as 0.00013 ng/mL (0.06 pM) were reached in the case of the lung cancer cell line NCI-H2122. In comparison to the parental IgG, the MB resulted in more than a 27,000-fold potency enhancement (in mass) and more than a 418,000-fold potency enhancement (in molar). In the case of the Conatumumab Multabody, similar IC₅₀ values were obtained across a cancer cell line panel. The parental Conatumumab IgG showed overall a higher potency than Tigatuzumab IgG.

TABLE 1

| | DR5 IC₅₀ (μg/mL) | | | | | |
|---|---|---|---|---|---|---|
| | Tigatuzumab | | Conatumumab | | Fold-change | |
| | IgG | MB | IgG | MB | Tigatuzumab | Conatumumab |
| H2122 | 0.4 | 0.00014 | 0.019 | 0.00015 | 2939 | 129 |
| MDA-BD231 | >10 | 0.0012 | 2.4 | 0.00071 | >8403 | 3380 |
| H2228 | >10 | 0.015 | >10 | 0.031 | >651 | >322 |
| Colo 205 | >10 | 0.00085 | 0.22 | 0.00037 | >11768 | 595 |
| SW948 | >10 | 0.00037 | 0.46 | 0.00037 | >27027 | 1243 |
| HT29 | >10 | 0.0084 | 2.2 | 0.0024 | >1190 | 917 |
| HTC15 | >10 | 0.0020 | 1.2 | 0.00053 | >5000 | 2264 |
| CAPAN-1 | >10 | 0.0026 | 1.9 | 0.0020 | >5000 | 950 |

| | DR5 IC₅₀ (nM) | | | | | |
|---|---|---|---|---|---|---|
| | Tigatuzumab | | Conatumumab | | Fold-change | |
| | IgG | MB | IgG | MB | Tigatuzumab | Conatumumab |
| H2122 | 2.7 | 0.000059 | 0.13 | 0.000064 | 45628 | 1971 |
| MDA-BD231 | >67 | 0.00052 | 16 | 0.00031 | >129495 | 51831 |
| H2228 | >67 | 0.0067 | >67 | 0.013 | >10045 | >4971 |
| Colo 205 | >67 | 0.00037 | 1.5 | 0.00016 | >181358 | 9117 |
| SW948 | >67 | 0.00016 | 3.1 | 0.00016 | >416486 | 19063 |
| HT29 | >67 | 0.0037 | 15 | 0.0010 | >18345 | 14056 |
| HTC15 | >67 | 0.00087 | 8 | 0.00023 | >77050 | 34717 |
| CAPAN-1 | >67 | 0.0011 | 13 | 0.00087 | >59269 | 14567 |

### Example 2: Therapeutic Effect of DR5-Targeting Multabodies in a Xenograft Mouse Model

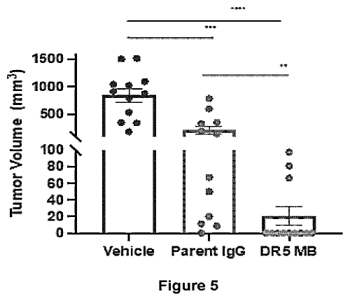

The therapeutic effect of exemplary Multabodies was evaluated in a colon cancer xenograft model. 5×10⁶ human colon cancer cells were injected subcutaneously in the flank of immunodeficient mice (n=12 per group). Mice with established tumors received treatment or vehicle control via intra-peritoneal (i.p.) injection once weekly for two weeks. Tumor volume was measured twice weekly using calipers. FIG. 5 shows the tumor volume at day 88 after study initiation. Treatment with DR5 MB significantly inhibited tumor growth of established tumors. DR5 MB inhibited tumor growth more strongly than DR5 IgG.

### SEQUENCE LISTING

Underlining within fusion sequences indicate linker sequences.
Bolding within fusion sequences indicate ferritin or ferritin subunit sequences.
Boxed and bolded residues indicate residues that are mutated relative to a reference molecule. e.g. relative to an IgG1 Fc.

hFTL
(SEQ ID NO: 56)
MSSQIRQNYSTDVEAAVNSLVNLYLQASYTYLSLGFYFDRDDVALEGVSHFFRELAEEKREG
YERLLKMQNQRGGRALFQDIKKPAEDEWGKTPDAMKAAMALEKKLNQALLDLHALGSARTDP
HLCDFLETHFLDEEVKLIKKMGDHLTNLHRLGGPEAGLGEYLFERLTLRHD
N_hFTL
(SEQ ID NO: 57)
MSSQIRQNYSTDVEAAVNSLVNLYLQASYTYLSLGFYFDRDDVALEGVSHFFRELAEEKREG
YERLLKMQNQRGGRALFQDIKKPAEDEW
C_hFTL
(SEQ ID NO: 58)
GKTPDAMKAAMALEKKLNQALLDLHALGSARTDPHLCDFLETHFLDEEVKLIKKMGDHLTNL
HRLGGPEAGLGEYLFERLTLRHD
IgG1 Fc
(SEQ ID NO: 59)
DKTHTCPPCPAPELLGGPSVFLFPPKPKDTLMISRTPEVTCVVVDVSHEDPEVKENWYVDGV
EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFEL
YSKLTVDKSRWQQGNVFSCSVMHEALHNHYTQKSLSLSPGK
IgG1 scFc
(SEQ ID NO: 60)
DKTHTCPPCPAPELLGGPSVFLFPPKPKDTLMISRTPEVTCVVVDVSHEDPEVKENWYVDGV
EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGGGGGSG
GGGSGGGGSGGGGSGGGGSGGGGSGGGGSGGGGSGGGGSGGGGSGGGGSDKTHTCPPCPAPE
LLGGPSVFLFPPKPKDTLMISRTPEVTCVVVDVSHEDPEVKENWYVDGVEVHNAKTKPREEQ
YNSTYRVVSVLIVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRWQQ
GNVFSCSVMHEALHNHYTQKSLSLSPGK
Cona LC
(SEQ ID NO: 61)
EIVLTQSPGTLSLSPGERATLSCRASQGISRSYLAWYQQKPGQAPSLLIYGASSRATGIPDR
FSGSGSGTDFTLTISRLEPEDFAVYYCQQFGSSPWTFGQGTKVEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSENRGEC
Cona HC
(SEQ ID NO: 62)
QVQLQESGPGLVKPSQTLSLTCTVSGGSISSGDYFWSWIRQLPGKGLEWIGHIHNSGTTYYN
PSLKSRVTISVDTSKKQFSLRLSSVTAADTAVYYCARDRGGDYYYGMDVWGQGTTVTVSSAS
TKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAPELLGGPSVELF
PPKPKDTLMISRTPEVTCVVVDVSHEDPEVKENWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
Cona scFab
(SEQ ID NO: 63)
EIVLTQSPGTLSLSPGERATLSCRASQGISRSYLAWYQQKPGQAPSLLIYGASSRATGIPDR
FSGSGSGTDFTLTISRLEPEDFAVYYCQQFGSSPWTFGQGTKVEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSFNRGECGGGGSGGGGSGGGGSGGGGSGGGGSGGGGSGGG
GSGGGGSGGGGSGGGGSGGGGSGGGGSGGGGSGGGGSQVQLQESGPGLVKPSQTLSLTCTVS
GGSISSGDYFWSWIRQLPGKGLEWIGHIHNSGTTYYNPSLKSRVTISVDTSKKQFSLRLSSV
TAADTAVYYCARDRGGDYYYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKP
SNTKVDKKVEPKSC

1. A fusion protein comprising a nanocage monomer or a subunit thereof linked to a DR4 and/or DR5 antigen-binding moiety, wherein a plurality of the fusion proteins self-assemble to form a nanocage.

2. The fusion protein of claim 1, wherein the DR4 and/or DR5 antigen-binding moiety targets the DR4 and/or DR5 ectodomain.

3. (canceled)

4. The fusion protein of claim 1, wherein the DR4 and/or DR5 antigen-binding moiety comprises an antibody or fragment thereof.

5. The fusion protein of claim 4, wherein the antibody or fragment thereof comprises a Fab fragment.

6-9. (canceled)

10. The fusion protein of claim 4, wherein the fusion protein comprises a DR5 antigen-binding moiety.

11-14. (canceled)

15. The fusion protein of claim 1, wherein the fusion protein comprises a first nanocage monomer subunit linked to the DR4 and/or DR5 antigen-binding moiety; wherein the first nanocage monomer subunit is capable of self-assembling with a second nanocage monomer subunit to form the nanocage monomer.

16-25. (canceled)

26. The fusion protein of claim 1, wherein the nanocage monomer is selected from the group consisting of ferritin, apoferritin, encapsulin, SOR, lumazine synthase, pyruvate dehydrogenase, carboxysome, vault proteins, GroEL, heat shock protein, E2P, MS2 coat protein, fragments thereof, and variants thereof.

27. The fusion protein of claim 26, wherein the nanocage monomer is apoferritin.

28. The fusion protein of claim 26, wherein the nanocage monomer is an apoferritin light chain.

29. The fusion protein of claim 27, wherein the fusion protein comprises a first apoferritin subunit and wherein the first apoferritin subunit is capable of self-assembling with a second apoferritin subunit.

30. The fusion protein of claim 29, wherein the first and second apoferritin monomer subunits interchangeably comprise the “N” and “C” regions of apoferritin.

31-32. (canceled)

33. The fusion protein of claim 1, further comprising a bioactive moiety and a linker between the nanocage monomer or subunit thereof and the bioactive moiety.

34. The fusion protein of claim 33, wherein the linker is flexible or rigid and comprises from about 1 to about 30 amino acid residues.

35. The fusion protein of claim 33, wherein the linker comprises at least one GGGGS repeat.

36. (canceled)

37. The fusion protein of claim 1, further comprising a C-terminal linker.

38-39. (canceled)

40. A nanocage comprising at least one fusion protein of claim 1 and at least one second nanocage monomer or subunit thereof that self-assembles with the fusion protein.

41. The nanocage of claim 40, wherein the fusion protein comprises a first nanocage monomer subunit, the second nanocage monomer or subunit thereof is a second nanocage monomer subunit, and the second nanocage monomer subunit self-assembles with the fusion protein to form the nanocage monomer.

42-46. (canceled)

47. The nanocage of claim 40, wherein at least one DR4 and/or DR5 antigen-binding moiety decorates the exterior surface of the nanocage and at least one Fc fragment decorates the exterior surface of the nanocage.

48. The nanocage of claim 47, wherein at least two DR4 and/or DR5 antigen-binding moieties decorate the exterior surface of the nanocage and at least two Fc fragments decorate the exterior surface of the nanocage.

49-53. (canceled)

54. The nanocage of claim 40, carrying a cargo molecule selected from the group consisting of a diagnostic agent, and an imaging agent.

55-60. (canceled)

61. A DR4 and/or DR5 therapeutic or prophylactic composition comprising the nanocage of claim 40.

62. A nucleic acid molecule encoding the fusion protein of claim 1.

63. A vector comprising the nucleic acid molecule of claim 62.

64. A host cell comprising the vector of claim 63.

65. A method for treating and/or preventing cancer, the method comprising administering to the subject the nanocage of claim 40.

66. The method of claim 65, wherein the cancer is selected from the group consisting of breast cancer, colon cancer, lymphoma, and lung cancer.

67-68. (canceled)
